(19) United States
(12) Patent Application Publication
(10) Pub. No.: US 2011/0202142 A1
(43) Pub. Date: Aug. 18, 2011

(54) BIOLOGICALLY DERIVED COMPOSITE TISSUE ENGINEERING

(75) Inventors: Jeremy J. Mao, Closter, NJ (US); Chang Hun Lee, New York, NY (US)

(73) Assignee: The Trustees of Columbia University in the City of New York, New York, NY (US)

(21) Appl. No.: 12/666,235

(22) PCT Filed: Jul. 2, 2008
(86) PCT No.: PCT/US08/69114

§ 371 Date: Feb. 11, 2011

Related U.S. Application Data
(60) Provisional application No. 60/947,642, filed on Jul. 2, 2007.

(51) Int. Cl. A61F 2/02

(52) U.S. Cl. 623/23.72

(57) ABSTRACT

The present application is directed to engineering of tissues, especially composite tissues such as a joint. Various aspects of the application provide tissue modules and methods of fabrication and use thereof. Some embodiments provide a tissue module that can be fabricated to be substantially similar in anatomic internal and external shape as a target tissue. Some embodiments provide a composite tissue module having a plurality of layers, each of which simulate a different tissue (e.g., bone and cartilage of a joint).

### CROSS-REFERENCE TO RELATED APPLICATIONS

This application claims the benefit of U.S. provisional application Ser. No. 60/947,642, filed Jul. 2, 2007, incorporated herein by reference in its entirety.

### STATEMENT REGARDING FEDERALLY SPONSORED RESEARCH OR DEVELOPMENT

This invention was made in part with Government support under National Institutes of Health Grants R01DE15391 and R01EB02332. The Government has certain rights in the invention.

### FIELD

The present application generally relates to tissue engineering, especially of composite tissue.

### BACKGROUND

A common roadblock in regeneration of traumatized or diseased human tissue or organs is scale up and associated challenges including vascularization, cell survival and functionality. Regenerating tissue over 100-200 μm generally exceeds the capacity of nutrient diffusion and waste removal, and thus requires vascular supply. A skeletal muscle graft (˜5×3 mm) was vascularized in vivo following co-seeding of myoblasts, mouse embryonic fibroblasts and endothelial cells. Collagen scaffolds (˜15×4 mm) with neonatal cardiac myoblasts engrafted into infarcted rat cardiac muscle, and improved cardiac function. A bioartificial heart was created ex vivo in a bioreactor by seeding cardiac or endothelial cells in decellularized rat hearts, and generated about 2% of adult heart function. Sheets of collagen and/or polyglycolite seeded with autologous mesenchymal stem cells successfully led to tissue-engineered bladders in seven patients. Strides have been made towards the tissue engineering of vascular grafts.

But a common aspiration by surgeons and scientific community to replace diseased or missing native tissue and organs with biological substitutes is yet to be broadly realized. The hypothesis that tissue regeneration is enhanced by bioengineered scaffolds remains to be rigorously demonstrated and tested in vivo (review: Hutmacher and Cool, 2007, J Cell Mol Med. 11(4):654-69).

A synovial joint is an organ consisting of multiple tissues including articular cartilage and subchondral bone. Skeletal motion in terrestrial mammals is accomplished by synovial joints. Osteoarthritis represents structural breakdown of cartilage and bone of the synovial joint, and is the leading cause of chronic disabilities worldwide, affecting approximately 80.8 million people in the United States alone (Kraus, 1997, Med Clin North Am 81:85-112; Lawrence et al., 1998, Arthritis Rheum 43:778-799; CDC 2001, MMWR 50:120-125). The cost of treating arthritis and related conditions in the U.S. alone is over 75 billion dollars per year (Lawrence et al., 1998, Arthritis Rheum 43:778-799; CDC 2001, MMWR 50:120-125). A somewhat modest goal towards biologically based therapies for osteoarthritis has been set to repair small, localized cartilage or osteochondral defects by means of bioengineered plugs. A number of studies have shown that cartilage-bone composite tissue can be regenerated in surgically created focal defects in synovial joints of several species (Gao et al., 2001, Tissue Eng 7:363-371). But commercial approaches to transplant cartilage plugs or chondrocytes to replace arthritic cartilage tissue in patients have only witnessed limited success (Wood et al., 2006, J Bone Joint Surg Am 88:503-507). Drawbacks of cartilage or osteochondral plugs such as suboptimal integration, loss of chondrocyte phenotype and guarded functional outcome have not been overcome (Wood et al., 2006, J Bone Joint Surg Am 88:503-507). Current approaches for cartilage injuries, including chondrocyte transplantation, cartilage grafts and artificial prostheses, suffer from deficiencies such as donor site morbidity, limited tissue supply, immunorejection, potential pathogen transmission, implant dislocation, wear and tear.

Clinically, localized cartilage lesions frequently deteriorate into more severe arthritis that warrants total joint arthroplasty (Caplan and Goldberg, 1999, Clin Orthop Relat Res. 367 Suppl:S12-6; Buckwalter and Martin, 2006, Adv Drug Deliv Rev 58(2):150-67). Accordingly, biologically based cartilage resurfacing or replacement of the entire synovial joint condyle has been proposed (Moutos et al., 2007, Nat Mater 6(2):162-7). It has been reported that stratified layers of cartilage and bone structures with dimensions of the entire human synovial joints can be regenerated ectopically in vivo from several cell sources such as a single population of bone marrow-derived mesenchymal stem cells (MSCs) (Alhadlaq et al., 2004, Stem Cell and Development 13:436-448), or differentiated chondrocytes and osteoblasts (Isogai et al., 1999, J Bone Joint Surg Am. 81:306-16; Weng et al., 2001, J Oral Maxillofac Surg 59:185-190). And previous studies have generated human-shaped synovial joint condyles ectopically.

But no previous work has achieved one of the important goals of orthopedic medicine to replace arthritic joint with a biologically based articulation in a weight-bearing environment.

Cell transplantation is the default strategy of cell based therapies, but has encountered several critical barriers within the current health care infrastructure. Technological and economic viability of cell transplantation approaches, especially those that require substantial cell manipulation ex vivo, has been questioned . Recently, there is growing interest to regenerate tissues by cell homing (Chamberlain et al., 2007, Stem Cells 25(11):2739-49; Laird et al., 2008, Cell 132(4):612-30). If successful, cell homing will attract host's native cells, including stem cells, to the anatomic location of trauma or diseases. The recruited host cells may then release signaling cues and/or participate in tissue healing. Conceptually, successful tissue regeneration by cell homing and without cell transplantation may overcome several critical barriers of cell-based therapies. But cell recruitment towards tissue regeneration, especially without cell transplantation, remains unproven.

### SUMMARY

Disclosed herein is a new approach towards the engineering of tissue modules, especially those composed of one or more types of tissues in various structural and functional arrangements. Tissue modules produced using the disclosed compositions and methods can be used in various clinical applications.

One provided aspect is directed to a tissue module comprising a biocompatible matrix comprising at least two layers, a first layer and a second layer, wherein the first matrix layer has a first plurality of internal microchannels with a first average diameter; and the second matrix layer has a second plurality of internal microchannels with a second average diameter. In some embodiments, the second matrix layer surrounds, at least in part, the first matrix layer.

In some embodiments, the tissue module includes a first type of progenitor cells seeded in the first matrix layer and a second type of progenitor cells seeded in the second matrix layer.

In some embodiments, the average microchannel diameter of the first matrix layer is the same as the average microchannel diameter of the second matrix layer. In some embodiments, the average microchannel diameter of the first matrix layer is approximately the same as the average microchannel diameter of the second matrix layer. In various embodiments, the average microchannel diameter of the first and/or second matrix is about 100 μm to about 600 μm. In various embodiments, the average microchannel diameter of the first and/or second matrix is about 100 μm, about 150 μm, about 200 μm, about 250 μm, about 300 μm, about 350 μm, about 400 μm, about 450 μm, about 500 μm, about 550 μm, or about 600 μm. In various embodiments, the average microchannel diameter of the first and/or second matrix is about 200 μm to about 400 μm.

In some embodiments, the average microchannel diameter of the first matrix layer is different than the average microchannel diameter of the second matrix layer. In various embodiments, the average microchannel diameter of the first matrix layer is about 100 μm to about 400 μm; the average microchannel diameter of the second matrix layer is about 200 μm to about 600 μm; and the average microchannel diameter of the first matrix layer is less than that of the second matrix layer. In various embodiments, the average microchannel diameter of the first matrix layer is about 100 μm, about 150 μm, about 200 μm, about 250 μm, about 300 μm, about 350 μm, or about 400 μm; the average microchannel diameter of the second matrix layer is about 200 μm, about 250 μm, about 300 μm, about 350 μm, about 400 μm, about 450 μm, about 500 μm, about 550 μm, or about 600 μm; and the average microchannel diameter of the first matrix layer is less than that of the second matrix layer. In various embodiments, the average microchannel diameter of the first matrix layer is about 200 μm; and the average microchannel diameter of the second matrix layer is about 400 μm.

In some embodiments, the matrix is an anatomically-shaped 3D composite biocompatible matrix comprising a plurality of interlaid strands forming internal microchannels.

In some embodiments, the first matrix layer and the second matrix layer comprise at least one material independently selected from the group consisting of fibrin, fibrinogen, a collagen, a polyorthoester, a polyvinyl alcohol, a polyamide, a polycarbonate, a polyvinyl pyrrolidone, a marine adhesive protein, a cyanoacrylate, and a polymeric hydrogel, or a combination thereof. In various embodiments, the first matrix layer and the second matrix layer comprise substantially the same material. In various embodiments, the first matrix layer and the second matrix layer comprise different materials. In various embodiments, the first matrix layer and/or the second matrix layer comprise polycaprolactone. In various embodiments, the first matrix layer and/or the second matrix layer further comprises hydroxyapatite. In various embodiments, the first matrix layer comprises polycaprolactone and the second matrix layer comprises polyethylene glycol hydrogel.

In some embodiments, the first type of progenitor cells are bone progenitor cells. In various embodiments, the bone progenitor cells are mesenchymal stem cells (MSC), MSC-derived cells, or osteoblasts, or a combination thereof. In various embodiments, the bone progenitor cells comprise MSCs. In various embodiments, the bone progenitor cells comprise osteoblasts.

In some embodiments, the second type of progenitor cells are cartilage progenitor cells. In various embodiments, the cartilage progenitor cells are esenchymal stem cells (MSC), MSC-derived cells, or chondrocytes, or a combination thereof. In various embodiments, the cartilage progenitor cells comprise MSCs. In various embodiments, the cartilage progenitor cells comprise chondrocytes.

In some embodiments, the tissue module comprises progenitor cells at a density of at least about 0.0001 million cells (M) ml⁻¹ up to about 1000 M ml⁻¹. In various embodiments, the tissue module comprises progenitor cells at a density of about 1 M ml⁻¹, about 5 M ml⁻¹, about 10 M ml⁻¹, about 15 M ml⁻¹, about 20 M ml⁻¹, about 25 M ml⁻¹, about 30 M ml⁻¹, about 35 M ml⁻¹, about 40 M ml⁻¹, about 45 M ml⁻¹, about 50 M ml⁻¹, about 55 M ml⁻¹, about 60 M ml⁻¹, about 65 M ml⁻¹, about 70 M ml⁻¹, about 75 M ml⁻¹, about 80 M ml⁻¹, about 85 M ml⁻¹, about 90 M ml⁻¹, about 95 M ml⁻¹, or about 100 M ml⁻¹. In various embodiments, the ratio of the first type of progenitor cells to the second type of progenitor cells is from at least about 100:1 up to about 1:100. In various embodiments, the ratio of the first type of progenitor cells to the second type of progenitor cells is about 20:1, about 19:1, about 18:1, about 17:1, about 16:1, about 15:1, about 14:1, about 13:1, about 12:1, about 11:1, about 10:1, about 9:1, about 8:1, about 7:1, about 6:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, or about 1:20.

In some embodiments, the first matrix layer and/or the second matrix layer further comprise at least one agent selected from the group consisting of a bioactive molecule, biologic drug, diagnostic agent, or strengthening agent; or the step of introducing an agent selected from the group consisting of a bioactive molecule, biologic drug, diagnostic agent, and strengthening agent to the matrix material, or a combination thereof. In various embodiments, the first matrix layer and/or the second matrix layer comprise at least one agent independently selected from the group consisting of an osteoinductive cytokine and a chondroinductive cytokine In various embodiments, the first matrix layer and/or the second matrix layer comprises at least one agent independently selected from an the group consisting of TGFβ, bFGF, VEGF, and PDGF, or a combination thereof. In various embodiments, the first matrix layer and/or the second matrix layer comprises TGFβ3.

In some embodiments, the first matrix layer and/or the second matrix layer comprise a plurality of pores having an average diameter of about 100 μm to about 600 μm. In various embodiments, the first matrix layer and/or the second matrix layer comprise a plurality of pores having an average diameter of about 100 μm, about 150 μm, about 200 μm, about 250 μm, about 300 μm, about 350 μm, about 400 μm, about 450 μm, about 500 μm, about 550 μm, or about 600 μm.

In some embodiments, the biocompatible matrix has a 3D anatomical shape selected from the group consisting of a fibrous joint, a cartilaginous joint, or a synovial joint. In various embodiments, the biocompatible matrix has a 3D anatomical shape of a synovial joint selected from the group consisting of a ball and socket joint, condyloid joint, saddle joint, hinge joint, pivot joint, and gliding joint. In various embodiments, the biocompatible matrix has a 3D anatomical shape of a synovial joint selected from the group consisting of a proximal tibial condyle, proximal humeral condyle, femoral condyle, and mandibular condyle.

Another provided aspect is a method of treating a tissue defect in a subject comprising grafting a tissue module described herein into a subject in need thereof. In some embodiments, the tissue defect is associated with arthritis; osteoarthritis; osteoporosis; osteochondrosis; osteochondritis; osteogenesis imperfecta; osteomyelitis; osteophytes; achondroplasia; costochondritis; chondroma; chondrosarcoma; herniated disk; Klippel-Feil syndrome; osteitis deformans; osteitis fibrosa cystica, a congenital defect resulting in absence of a tissue; accidental tissue defect; fracture; wound; joint trauma; an autoimmune disorder; diabetes; cancer; a disease, disorder, or condition that requires the removal of a tissue; and/or a disease, disorder, or condition that affects the trabecular to cortical bone ratio. In some embodiments, the subject is a mammal, reptile, or avians. In some embodiments, the subject is a horse, cow, dog, cat, sheep, pig, or chicken. In some embodiments, the subject is a human.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a series of images showing a human-shaped proximal tibial condyle of the knee joint engineered from polycaprolactone (PCL), a biodegradable polymeric material that simulates the mechanical properties of bone. Pores and channels with a diameter of 400 um were designed by computer-aided design (CAD) and fabricated with a Bioplotter via computer-aided manufacturing (CAM) approach. Cells and/or growth factors were deposited in the PCL. FIG. 1A presents a side view of the engineered joint. FIG. 1B presents a superior view of the engineered joint. FIG. 1C presents an inferior view of the engineered joint. For details regarding methodology, see Example 1.

FIG. 2 is a series of images showing a human-shaped femoral condyle of the hip joint engineered from polycaprolactone (PCL), a biodegradable polymeric material that simulates the mechanical properties of bone. Pores and channels with a diameter of 400 um were designed by computer-aided design (CAD) and fabricated with a Bioplotter via computer-aided manufacturing (CAM) approach. FIG. 2A presents a posterior side view of the engineered joint. FIG. 2B presents an anterior side view of the engineered joint. FIG. 2C presents a superior view of the engineered joint. FIG. 2D presents an inferior view of the engineered joint. For details regarding methodology, see Example 2.

FIG. 3 is a series of images showing a human-shaped mandibular condyle of the temporomandibular joint engineered from polycaprolactone (PCL), a biodegradable polymeric material that simulates the mechanical properties of bone. Pores and channels with a diameter of 400 um were designed by computer-aided design (CAD) and fabricated with a Bioplotter via computer-aided manufacturing (CAM) approach. Cells and/or growth factors were deposited in the PCL. FIG. 3A presents a side view of the engineered joint. FIG. 3B presents a superior view of the engineered joint. FIG. 3C presents an oblique view of the engineered joint. For details regarding methodology, see Example 3.

FIG. 4 is a series of images showing a human-shaped proximal tibia condyle of the knee joint engineered from two composite materials, a hydrogel material that is anchored to a stiff material. Hydrogel material simulates articular cartilage, whereas stiff material stimulates subchondral bone. The hydrogel material used in this example is polyethylene glycol (PEG) hydrogel used for cartilage regeneration. The stiff material is polycaprolactone (PCL), a biodegradable polymeric material that simulates the mechanical properties of bone. A thin layer of PEG hydrogel 1-2 mm was anchored the pores and channels of the PCL. Chondrocytes or stem cell-derived chondrocytes were seeded in PEG hydrogel, whereas osteoblasts or stem cell-derived osteoblasts were seeded in PCL. Growth factors were deposited in the PCL. Pores and channels can be used for seeding cells and/or growth factors, or serve as conduits for vascularization. For details regarding methodology, see Example 4.

FIG. 5 is a series of showing a human-shaped proximal tibia condyle of the knee joint engineered from two composite materials, a thin (1-2 mm) layer of polyethylene glycol (PEG) hydrogel anchored to polycaprolactone (PCL), that were harvested from nude rat following 4 week in vivo implantation. MSC derived chondrocytes were seeded in PEG hydrogel, whereas MSC-derived osteoblasts were seeded in PCL. FIG. 5A shows the overall shape and two layers of cartilage and bone of the harvested engineered joint. FIG. 5B shows the porosity of the of the harvested engineered joint. FIG. 5C shows an H&E section of the osteochondral interface in the central region of a (box with solid line) showing cells populated both the cartilage layer (solid arrow) and bone layer (dashed arrow), with visible areas of vascularization. FIG. 5D shows an H&E section of the osteochondral interface in the peripheral region (dashed box in a) showing cortical like structure populated with cells, as well as cartilage hydrogel layer populated with cells. For details regarding methodology, see Example 5.

FIG. 6 is a series of images and schematic diagrams showing Bioengineering design and surgical replacement of a rabbit shoulder joint. FIG. 6A shows the 3D anatomic contour of a cadaver proximal humeral condyle of a skeletally mature rabbit captured with multi-slice laser scanning at a resolution of 100 μm. FIG. 6B shows an anatomically shaped scaffold (darker gray) with a retention stem designed from the 3D anatomic contour. FIG. 6C is a schematic diagram of dimensional parameters of the engineering design of the anatomically shaped scaffold with retention stem. FIG. 6D is a an image of the articular surface of a 200-μm thick shell with interconnecting micropores and microchannels that open in both articular surface and bone marrow surface. FIG. 6E is an image of the bone marrow surface of a 200-μm thick shell with interconnecting micropores and microchannels that open in both articular surface and bone marrow surface. Poly E-caprolactone (PCL) and hydroxyapatite (HA) were comelted into slurry and fabricated into anatomical shape and dimensions of the scaffold (FIG. D, FIG. E) by following the engineering design (FIG. C). FIG. 6F is an image of an operation where an osteotome was removed from the right proximal humeral condyle at 5 mm depth from articular surface. FIG. 6G is an image of an orthopedic drill being used to prepare subchondral bone for insertion of the stem (see FIG. 6B) into marrow cavity. FIG. 6H is an image showing the excised condylar head (top left) and the engineered anatomically shaped scaffold (bottom right). FIG. 6I is an image showing the like-shaped, bioengineered condylar head replacement secured by inserting the stem into bone marrow cavity and secured by press-fit. Scale: 400 μm. For details regarding methodology, see Examples 6-7.

FIG. 7 is a series of images and bar graphs showing regeneration of cartilage and subchondral bone in bioengineered joint scaffolds after retrieval of in vivo implanted joint replacement constructs at 8 and 16 wks post-op. FIG. 7A is an image of an un-implanted scaffold sample. FIG. 7B is an image of an articular surface formed per Indian Ink in TGFβ3-free samples. FIG. 7C is an image of an articular surface formed per Indian Ink in TGFβ3-loaded samples. FIG. 7D is an image of a native articular surface. FIG. 7E is an image of chondrocyte-like cells from TGFβ3-free samples labeled with Saf-O. FIG. 7F is an image of chondrocyte-like cells, pericellular matrix, and in terterritorial matrix from TGFβ3-free samples labeled with Saf-O. FIG. 7G is an image of chondrocyte-like cells from TGFβ3-loaded samples labeled with Saf-O. FIG. 7H is an image of chondrocyte-like cells, pericellular matrix, and in terterritorial matrix from TGFβ3-loaded samples labeled with Saf-O. FIG. 7I is a bar graph showing cartilage density for TGFβ3-free samples and TGFβ3-loaded samples. FIG. 7J is a bar graph showing cartilage thickness (μm) for TGFβ3-free samples and TGFβ3-loaded samples. Scale: 100 μm. For details regarding methodology, see Example 8.

FIG. 8 is a series of images and bar graphs showing TGFβ3 delivery improves engineered cartilage matrix. FIG. 8A is a series of images showing immunoblotting with monoclonal antibodies for type II collagen (Col-11) (left column) and aggrecan (AGC) (right column) of the in +TGFβ3 samples, −TGFβ3 samples, and native samples with an articular surface view. FIG. 8B is a series of images showing immunoblotting with monoclonal antibodies for type II collagen (Col-11) (left column) and aggrecan (AGC) (right column) of the in +TGFβ3 samples, −TGFβ3 samples, and native samples with a sagittal section view. FIG. 8C is a bar graph showing Col-II immunoreactivity for native samples (left bar), −TGFβ3 samples (middle bar), and +TGFβ3 samples (right bar) in articular surface (left grouping of bars) and sagittal section (right grouping of bars). n=10 per group, #:p=0.0329, +:p=0.00001, *:p=0.0035, ##:p=0.015, ++:p=0.0001, **:p=0.038. FIG. 8D is a bar graph showing AGC immunoreactivity for native samples (left bar), −TGβ3 samples (middle bar), and +TGFβ3 samples (right bar) in articular surface (left grouping of bars) and sagittal section (right grouping of bars). n=10 per group, #:p=0.037, +:p=0.00001, *:p=0.00292, ##:p=0.029, ++:p=0.0001, **:p=0.038. For details regarding methodology, see Example 8.

FIG. 9 is a series of images showing bioengineered subchondral bone integrates to bioengineered articular cartilage and host bone. FIG. 9A is an image showing radiolucency in the joint cavity of the excised condylar head in which bioengineered scaffold was implanted at Day 0. FIG. 9B is an image at 8 weeks post-op of the convex, radio-opaque condyle shaped structure present in the same rabbit that received the bioengineered scaffold. FIG. 9C is an image 16 weeks post-op of the convex, radio-opaque condyle shaped structure present in the same rabbit that received the bioengineered scaffold. FIG. 9D is an image showing bioengineered articular cartilage integrated to subchondral bone. FIG. 9E is an image showing bone trabecula-like structures in the subchondral bone. FIG. 9F is an image showing Von kossa staining of mineral deposition in microchannels that extends below the cartilage region (medium gray in FIG. 9F; see also FIG. 9C) longitudinally in microchannels. FIG. 9G is an image showing mineral apposition on the surface of PCL-HA strands that formed the wall of microchannels. FIG. 9H is an image showing bone trabeculae populated by columnar shaped osteoblast-like cells. FIG. 9I is an image bioengineered subchondral bone integrated to native humeral bone, with PCL-HA in the bioengineered bone above the dashed line, but native bone trabeculae, devoid of PCL-HA, below the dashed line. FIG. 9J and FIG. 9K are images showing multiple blood vessels present within microchannels with average vessel diameter of 67.11+/−28.35 μm. For details regarding methodology, see Example 9.

### DETAILED DESCRIPTION

The present application is directed towards engineered tissue modules and methods for their fabrication and use.

The present application is based, at least in part, on the successful replacement of shoulder joints in an animal model with anatomically shaped biomatrix scaffolds fabricated with repeating units of internal strands and microchannels, which allowed resumed locomotion and weight-bearing with all four limbs following surgery, along with regeneration of articular cartilage and subchondral bone (with osteoblast-populated bone trabeculae supplied by blood vessels) that is mineralized, vascularized and integrated with host bone. Given that no cells were transplanted, all regenerating cartilage and bone was determined to be host-derived. Bioengineered joint replacement has implications in treating arthritic or traumatized joints, and can regenerate large, complex tissues via cell homing.

Various aspects of the present application provide for inducing cartilage regeneration and synovial joint tissue engineering in biocompatible matrix materials in optional combination with seeded progenitor cells. The compositions, including engineered cartilage and/or bone, and methods described herein can be used to treat subjects with, for example, cartilage injuries, chronic diseases such as arthritis, joint trauma, and/or tumor resection. Thus is provided cartilage regeneration and/or synovial joint replacement compositions and procedures with improved efficacy, quality and/or life span.

Various approaches described herein can be used to fabricate custom-made biomaterial matrix with a pre-defined external shape and optional internally built-in channels that can serve as, for example, conduits for vascularization. The biomaterial matrix can be composed of composite biomaterials, where one material simulates one tissue type while another material simulates another tissue type. For example, various embodiments provide a composite tissue module having at least two matrix layers. In some configurations, one matrix layer can simulate cartilage and another bone. Optionally, cells can be introduced into the matrix. Cells introduced to the matrix can be progenitor cells, such as stem cells, so as to form the target tissue(s) being modeled.

Engineered Tissue

Various aspects of the application provide for tissue modules composed of a biocompatible matrix material, having one or more layers. In some embodiments, tissue modules composed of a biocompatible matrix material have one or more types of tissue progenitor cells incorporated therein. Some embodiments provide a composite tissue module having at least two matrix layers. These multiple matrix layers can simulate various cell or tissue types that combine to form a composite tissue. Tissues from which can be modeled the tissue modules described herein, include both hard and soft tissues. For example, the composite tissue module can have a similar, substantially the same, or the same shape and/or function of a biological hard tissue, such as cartilage, bones, and/or joints. Especially suitable tissues are those with a composite structure.

The methods and compositions described herein can be utilized to fabricate replacement joints. The tissue modules described herein can be modeled after a variety of joints including, but not limited to, fibrous joints (e.g., syndesmosis, somphosis, and sutures), cartilaginous joints (e.g., synchondroses such as the joint between the first rib and the manubrium of the sternum, and symphyses such as intervertebral discs and the pubic symphysis), and synovial joints. A tissue module modelled after a joint can be configured to include, for example, bone and cartilage (e.g., hyaline, elastic and/or fibrocartilage) in appropriate matrix layers.

The materials and methods described herein can be used to form engineered tissue modules, such as a synovial joint. Synovial joints (or diarthroses, diarthroidal joints) have a space between the articulating bones for synovial fluid. Synovial joints, such as the knee and shoulder, are generally the most mobile of the various joints. Synovial joints can be classified into ball and socket joints, condyloid joints (or ellipsoidal joints) (e.g., wrist), saddle joints (e.g., thumb), hinge joints (e.g., elbow, between the humerus and the ulna), pivot joints (e.g., elbow, between the radius and the ulna), and gliding joints (e.g., carpals of wrist). The bone surface at the joint is generally covered in cartilage. Various embodiments of the hard tissue modules described herein can mimic this cartilage layer with a thin, soft, pliable matrix material (optionally seeded with cartilage derivative cells), while an inner matrix material that is thicker and stiffer (and optionally seeded with bone progenitor cells) mimics the bone layer.

A hard tissue module can be modeled after, for example, a synovial joint such as the hip joint, the knee joint, the elbow joint, the phalanges, the temporomandibular joint, or a portion or component thereof. For example, the imaged hard tissue can be the proximal tibial condyle of the knee joint (see e.g., Examples 1, 4, and 5), the proximal humeral condyle (see e.g., Example 6), the femoral condyle of the hip joint (see e.g., Example 2), or the mandibular condyle of the temporomandibular joint (see e.g., Example 3).

The methods and compositions described herein can be utilized to fabricate replacement components of a vertebral column (i.e., backbone or spine). For example, one or more hard tissue modules can be modeled after vertebrae, sacrum, invertebral discs, and/or coccyx of a vertebral column. Vertebrae that can be modeled include cervical vertebrae (e.g., C1-C7), thoracic vertebrae (e.g., T1-T12), lumbar vertebrae (L1-L5), sacral vertebrae (S1-S5), and coccygeal vertebrae (e.g, Co1-Co4). In one example, a tissue module can be formed to simulate the complex of the coccyx (including from one to five segments), connecting fibrocartilaginous joint, and the sacrum (including from four to six segments) of a subject. As an alternative example, a tissue module can be formed to simulate one or more of these components.

Progenitor Cells

Various embodiments of methods and compositions described herein employ progenitor cells. The progenitor cell is generally of a type that can give rise to the target tissue(s) of interest. For example, when fabricating a replacement joint, which is composed of bone and cartilage, the progenitor cells of the tissue module can be bone progenitor cells and/or cartilage progenitor cells.

Progenitor cells can be isolated, purified, and/or cultured by a variety of means known to the art (see e.g., Example 4). Methods for the isolation and culture of progenitor cells are discussed in, for example, Vunjak-Novakovic and Freshney (2006) Culture of Cells for Tissue Engineering, Wiley-Liss, ISBN 0471629359. In some embodiments, progenitors cells can be from the same subject into which the tissue module is, or is to be, grafted. In other embodiments, progenitor cells can be derived from the same or different species as an intended transplant subject. For example, progenitor cells can be derived from an animal, including, but not limited to, a vertebrate such as a mammal, a reptile, or an avian. In some configurations, a mammal or avian is preferably a horse, a cow, a dog, a cat, a sheep, a pig, or a chicken, and most preferably a human.

Tissue progenitor cells of the present teachings include cells capable of differentiating into a target tissue, and/or undergoing morphogenesis to form the target tissue. Non-limiting examples of tissue progenitor cells include mesenchymal stem cells (MSCs), cells differentiated from MSCs, osteoblasts, chondrocytes, and fibroblastic cells such as interstitial fibroblasts, tendon fibroblasts, dermal fibroblasts, ligament fibroblasts, periodontal fibroblasts such as gingival fibroblasts, and craniofacial fibroblasts.

For example, in a composite joint tissue modules of some embodiments, tissue progenitor cells introduced into a matrix can be progenitor cells that can give rise to bone tissue such as mesenchymal stem cells (MSC) or MSC osteoblasts. It is understood that MSC osteoblasts are osteoblasts differentiated from MSC osteoblasts.

As another example, in various embodiments of a composite joint tissue modules, tissue progenitor cells introduced into a matrix can be progenitor cells that can give rise to cartilage tissue such as MSCs or MSC chondrocytes. It is understood that MSC chondrocytes are chondrocytes differentiated from MSCs. In various configurations, the cartilage progenitor cells can form hyaline cartilage, elastic cartilage, and/or fibrocartilage so as to approximate the structure and function of the target tissue being modeled.

It is understood that various types of progenitor cells can be seeded into the same matrix layer or each type into different matrix layers.

In some embodiments, the progenitor cells introduced to the matrix can comprise a heterologous nucleic acid so as to express a bioactive molecule such as heterologous protein, or to overexpress an endogenous protein. In non-limiting example, progenitor cells introduced to the matrix can express a fluorescent protein marker, such as GFP, EGFP, BFP, CFP, YFP, or RFP. In another example, progenitor cells introduced to the matrix can express an angiogenesis-related factor, such as activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation sphingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin and fibronectin receptor α₅β₁, Factor X, HB-EGF, HBNF, HGF, HUAF, heart derived inhibitor of vascular cell proliferation, IFN-gamma, IL1, IGF-2 IFN-gamma, integrin receptors (e.g., various combinations of α subunits (e.g., α₁, α₂, α₃, α₄, α₅, α₆, α₇, α₈, α₉, α_{E}, α_{V}, α_{IIb}, α_{L}, α_{M}, α_{X}), K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β3, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-beta, Tie 1, Tie2, TGF-β, and TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF.sub.164, VEGI, EG-VEGF, VEGF receptors, PF4, 16 kDa fragment of prolactin, prostaglandins E1 and E2, steroids, heparin, 1-butyryl glycerol (monobutyrin), or nicotinic amide. As another example, progenitor cells introduced to a matrix can comprise genetic sequences that reduce or eliminate an immune response in the host (e.g., by suppressing expression of cell surface antigens such as class I and class II histocompatibility antigen).

Matrix

Various compositions and methods of the application employ a matrix. In some embodiments, progenitor cells are introduced into or onto the matrix so as to form a tissue module. In various embodiments, the matrix materials are formed into a 3-dimensional scaffold. The scaffold can contain one or more matrix layers. For example, the scaffold can contain at least two matrix layers, at least three matrix layers, at least four matrix layers, at least five matrix layers, or more. Preferably, the scaffold contains two matrix layers. In some embodiments, the second matrix layer can cover and/or surround, at least in part, the first matrix layer.

The matrix and/or scaffold can: provide structural and/or functional features of the target tissue (e.g., bone and cartilage of a joint); allow cell attachment and migration; deliver and retain cells and biochemical factors; enable diffusion of cell nutrients and expressed products; and/or exert certain mechanical and biological influences to modify the behavior of the cell phase. The matrix materials of various embodiments are biocompatible materials that generally form a porous, microcellular scaffold, which provides a physical support and an adhesive substrate for introducing progenitor cells during in vitro fabrication and/or culturing and subsequent in vivo implantation.

A matrix with a high porosity and an adequate pore size is preferred so as to facilitate cell introduction and diffusion throughout the whole structure of both cells and nutrients. Matrix biodegradability is also preferred since absorption of the matrix by the surrounding tissues (e.g., after differentiation and growth of bone and cartilage tissues from progenitor cells) can eliminate the necessity of a surgical removal. The rate at which degradation occurs should coincide as much as possible with the rate of tissue formation. Thus, while cells are fabricating their own natural structure around themselves, the matrix can provide structural integrity and eventually break down leaving the neotissue, newly formed tissue which can assume the mechanical load. Injectability is also preferred in some clinical applications. Suitable matrix materials are discussed in, for example, Ma and Elisseeff, ed. (2005) Scaffolding in Tissue Engineering, CRC, ISBN 1574445219; Saltzman (2004) Tissue Engineering: Engineering Principles for the Design of Replacement Organs and Tissues, Oxford ISBN 019514130X.

The matrix configuration can be dependent on the tissue or organ that is to be repaired or produced. Preferably the matrix is a pliable, biocompatible, porous template that allows for target tissue growth. The matrix can be fabricated into structural supports, where the geometry of the structure (e.g., shape, size, porosity, micro- or macro-channels) is tailored to the application. The porosity of the matrix is a design parameter that influences cell introduction and/or cell infiltration. The matrix can be designed to incorporate extracellular matrix proteins that influence cell adhesion and migration in the matrix.

Preferably, at least two matrix materials are used to fabricate a tissue module described herein. The at least two matrix materials can be homogenously mixed throughout the scaffold, heterologously mixed throughout the scaffold, or separated into different matrix layers of the scaffold.

Matrices can be produced from proteins (e.g. extracellular matrix proteins such as fibrin, collagen, and fibronectin), polymers (e.g., polyvinylpyrrolidone), polysaccharides (e.g. alginate), hyaluronic acid, or analogs, mixtures, combinations, and derivatives of the above.

The matrix can be formed of synthetic polymers. Such synthetic polymers include, but are not limited to, poly(ethylene) glycol, bioerodible polymers (e.g., poly(lactide), poly(glycolic acid), poly(lactide-co-glycolide), poly(caprolactone), polyester (e.g., poly-(L-lactic acid), polyanhydride, polyglactin, polyglycolic acid), polycarbonates, polyamides, polyanhydrides, polyamino acids, polyortho esters, polyacetals, polycyanoacrylates), polyphosphazene, degradable polyurethanes, non-erodible polymers (e.g., polyacrylates, ethylene-vinyl acetate polymers and other acyl substituted cellulose acetates and derivatives thereof), non-erodible polyurethanes, polystyrenes, polyvinyl chloride, polyvinyl fluoride, polyvinyl pyrrolidone, poly(vinylimidazole), chlorosulphonated polyolifins, polyethylene oxide, polyvinyl alcohol (e.g., polyvinyl alcohol sponge), synthetic marine adhesive proteins, teflon®, nylon, or analogs, mixtures, combinations (e.g., polyethylene oxide-polypropylene glycol block copolymer; poly(D,L-lactide-co-glycolide) fiber matrix), and derivatives of the above.

The matrix can be formed of naturally occurring polymers or natively derived polymers. Such polymers include, but are not limited to, agarose, alginate (e.g., calcium alginate gel), fibrin, fibrinogen, fibronectin, collagen (e.g., a collagen gel), gelatin, hyaluronic acid, chitin, and other suitable polymers and biopolymers, or analogs, mixtures, combinations, and derivatives of the above. Also, the matrix can be formed from a mixture of naturally occurring biopolymers and synthetic polymers.

The matrix, or various matrix layers, can comprise a crystalline and/or mineral component. For example, the matrix, or various matrix layers, can include the inorganic mineral hydroxyapatite (also known as hydroxylapatite). About seventy percent of natural bone is made up of hydroxyapatite. In some embodiments, the matrix, or various matrix layers, comprises a ground natural substance containing hydroxyapatite, such as bone or dentin. In some embodiments, the matrix, or various matrix layers, comprises substantially pure hydroxyapatite

The matrix can comprise a composite matrix material comprising at least two components described above. As an example, a composite matrix material can comprise at least three, at least four, at least five, at least six, at least seven, at least eight, at least nine, at least ten, or more, components. The plurality of components can be homogenously mixed throughout the scaffold, heterologously mixed throughout the scaffold, or separated into different matrix layers of the scaffold, or a combination thereof.

A preferred matrix material is a composite matrix material comprising polycaprolactone and hydroxyapatite. In some embodiments, the matrix material comprises about 80 wt % polycaprolactone and about 20 wt % hydroxyapatite. In other embodiments, the matrix material comprises about 60 wt % polycaprolactone and about 40 wt % hydroxyapatite to about 95 wt % polycaprolactone and about 5 wt % hydroxyapatite. For example, the matrix material can comprise about 70 wt % polycaprolactone and about 30 wt % hydroxyapatite. As another example, the matrix material can comprise about 90 wt % polycaprolactone and about 10 wt % hydroxyapatite. In some embodiments, all matrix layers comprise polycaprolactone and hydroxyapatite. In other embodiments, a plurality of matrix layers comprise polycaprolactone and hydroxyapatite. In further embodiments, one matrix layer comprises polycaprolactone and hydroxyapatite.

Various embodiments of the application provide for a composite tissue module composed of two or more layers, where at least one layer comprises a matrix material suitable for serving as a bone tissue scaffold. Preferably, the bone tissue scaffold layer forms the core or central portion of the composite tissue module. The matrix material of the bone tissue scaffold should simulate the mechanical properties of bone. Examples of matrix material of the bone tissue scaffold are polycaprolactone (PCL) and polyethylene oxide. A preferred matrix material of the bone tissue scaffold is PCL.

In some embodiments, one or more matrix materials are modified so as to increase biodegradability. For example, PCL is a biodegradable polyester by hydrolysis of its ester linkages in physiological conditions, and can be further modified with ring opening polymerization to increase its biodegradability.

Various embodiments of the application provide for a composite tissue module composed of two or more layers, where at least one layer comprises a matrix material suitable for serving as a cartilage tissue scaffold. Preferably, the cartilage tissue scaffold layer forms an outer or outermost layer surrounding at least a portion of the composite tissue module. An example of a preferred matrix material of the cartilage tissue scaffold is PEG hydrogel.

In configurations having multiple layers of dissimilar matrix materials, one or more of the layers can provide structural characteristics that minimize or prevent disadhesion of one layer from another layer; functional integration of one layer with another layer; and/or dissociation of progenitor cells (or progeny thereof) from respective layers. For example, in embodiments having at least one matrix layer that simulates bone and at least one matrix layer that simulates cartilage, the porosity and/or channels of the “bone” matrix layer can minimize or prevent disadhesion of the “cartilage” matrix layer. Similarly, porosity and/or channels of the “bone” matrix layer and/or the “cartilage” matrix layer can minimize or prevent disassociation of progenitor cells and/or progeny cells therefrom.

Pores and Channels

Various embodiments of the application provide for a tissue module in which the matrix material of the fabricated scaffold contains pores and/or channels.

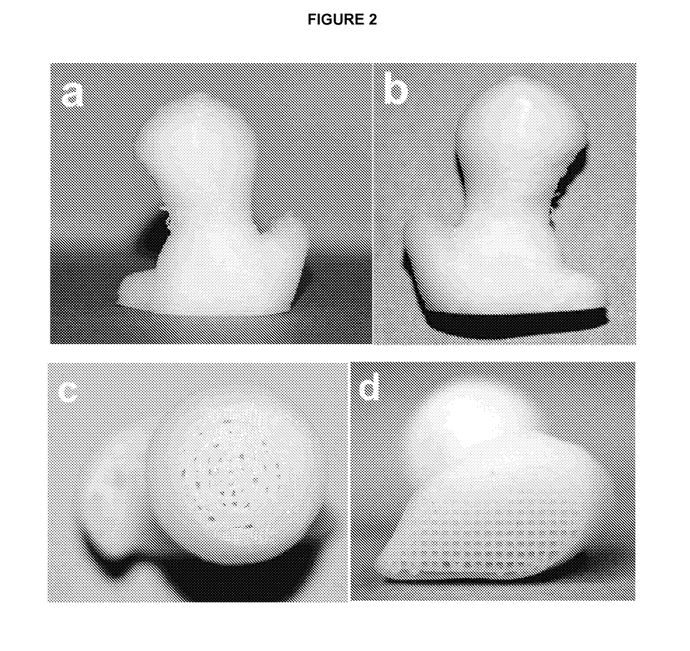

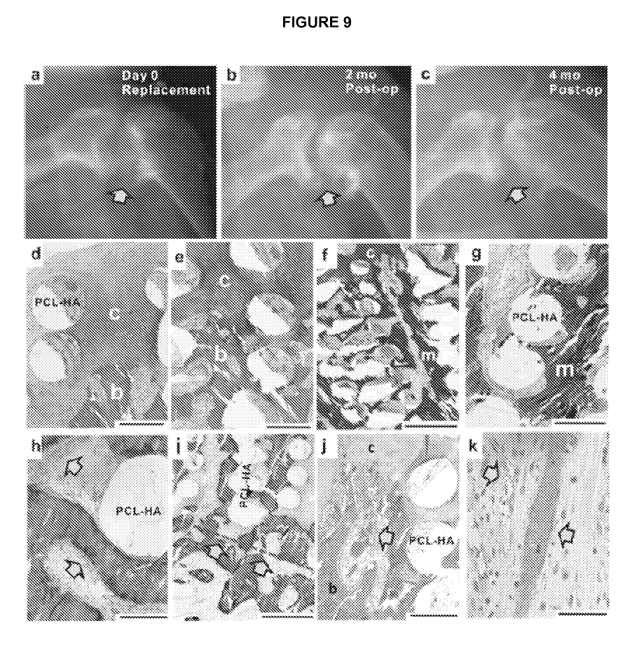

The pores of the scaffold can mimic internal bone structure, allow adherence of cells, provide an open volume for seeding of cells, provide an open volume for growth factors or other additives, allow adherence of another matrix layer, serve as conduits for vascularization, provide internal bone features, and/or facilitate perfusion. For example, internal pores of the matrix material of the scaffold can be configured to simulate bone trabeculae and the outer layer of the matrix material of the scaffold can be configured to simulate cortical bone (see e.g., Example 2, FIG. 2). As another example, internal pores of a composite-mixed scaffold can be configured to simulate bone trabecula-like structures (see e.g., Example 9; FIG. 9E).

Pores and channels of the matrix material can be engineered to be of various diameters. For example, the pores of the matrix material can have a diameter range from micrometers to millimeters. Preferably, the pores of the matrix material have a diameter of about 100 μm to about 600 μm (e.g., about 150 μm, about 200 μm, about 250 μm, about 300 μm, about 350 μm, about 400 μm, about 450 μm, about 500 μm, or about 550 μm). More preferably, the pores of the matrix material have a diameter of about 400 μm.

It is understood that the pores of the matrix material can have the same, approximately the same, or different average diameters between differing matrix layers of a scaffold. For example, a first matrix layer can have a first average pore diameter, a second matrix layer can have a second average pore diameter, and the first average pore diameter can be the same, approximately the same, or different than the second average pore diameter.

The matrix can contain one or more physical channels. Such physical channels include microchannels and macrochannels.

Microchannels generally have an average diameter of about 0.1 μm to about 1,000 μm. Preferably, microchannels have an average diameter of about 100 μm to about 600 μm (e.g., about 150 μm, about 200 μm, about 250 μm, about 300 μm, about 350 μm, about 400 μm, about 450 μm, about 500 μm, or about 550 μm), more preferably about 200 μm to about 400 μm. On skilled in the art will understand that the distribution of microchannel diameters can be a normal distribution of diameters or a non-normal distribution diameters. In some embodiments, microchannels are a naturally occurring feature of the matrix material(s). In some embodiments, microchannels are engineered to occur in the matrix materials.

In some embodiments, the engineered tissue module can have different average diameter microchannels in different portions of the construct. It is understood that the microchannels of the matrix material can have the same, approximately the same, or different average diameters between differing matrix layers of a scaffold. For example, a first matrix layer can have a first average microchannel diameter, a second matrix layer can have a second average microchannel diameter, and the first average microchannel diameter can be the same, approximately the same, or different than the second average microchannel diameter. In one embodiment, microchannels of a first average diameter can occur in a first region of the matrix while microchannels of a second average diameter can occur in a second region of the matrix. In some embodiments, the first average diameter of the first plurality of internal microchannels is about 100 μm to about 400 μm and the second average diameter of the second plurality of internal microchannels is about 200 μm to about 600 μm, with the first average diameter less than the second average diameter. In some embodiments, the first average diameter of the first plurality of internal microchannels is about 100 μm, about 150 μm, about 200 μm, about 250 μm, about 300 μm, about 350 μm, or about 400 μm; and the second average diameter of the second plurality of internal microchannels is about 200 μm, about 250 μm, about 300 μm, about 350 μm, about 400 μm, about 450 μm, about 500 μm, about 550 μm, or about 600 μm; where the first average diameter is less than the second average diameter. In one embodiments, the first average diameter of the first plurality of internal microchannels is about 200 μm; and the second average diameter of the second plurality of internal microchannels is about 400 μm.

As an example, interconnected microchannels with an average size of about 200 μm can occur throughout the scaffold except for the top layers down to about 1 mm, in which region occurs microchannels with an average size of about 400 μm. As another example, average microchannel diameter can be about 400 μm in the cartilage-like portion of the construct and about 200 μm in the bone-like portion of the construct.

In various embodiments, bioengineered scaffolds are modularizing with about 200 μm and about 400 μm repeat units of strands and inter-strand microchannels. One rationale for about 400 μm inter-strand microchannels in articular cartilage is that cartilage is devoid of vascular supply, whereas 200 μm inter-strand microchannels can be sufficient for generating vascularized subchondral bone in vivo.

Microchannels can facilitate and/or augment nutrient diffusion and waste removal. In some embodiments, microchannels can serve as a delivery channel and/or storage reservoir for additional components, such as active biological agents. In some embodiments, growth hormones can be introduced to the construct via microchannels. For example, TGFβ3 delivered in a collagen gel can be infused into scaffold microchannels followed by optional crosslinking gelation (see e.g., Example 7).

Matrix macrochannels can accelerate angiogenesis and bone or cartilage tissue formation, as well as direct the development of vascularization and host cell invasion. Macrochannels can be a naturally occurring feature of certain matrix materials and/or specifically engineered in the matrix material. Formation of macrochannels can be according to, for example, mechanical and/or chemical means.

To provide for enhanced vascularization, the matrix portion of the construct can be engineered to contain macrochannels. Constructs with engineered macrochannels can induce host tissue infiltration with vascular characteristics. Thus, tunnels, or similar structures, can be fabricated in the scaffold of the tissue module. Similar to the discussion regarding microchannels, macrochannel average diameters in different regions and/or matrix layers of the scaffold can be the same, approximately the same, or different.

Macrochannels can extend variable depths through the matrix material of the tissue module, or completely through the matrix material of the tissue module. Macrochannels can be a variety of diameters. Generally, the diameter of the macrochannel can be chosen according to increased optimization of perfusion, bone growth, cartilage growth, and vascularization of the tissue module. The macrochannels can have an average diameter of, for example, about 0.1 mm to about 50 mm. For example, macrochannels can have an average diameter of about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, about 10 mm, about 15 mm, about 20 mm, about 25 mm, about 30 mm, about 35 mm, about 40 mm, or about 45 mm. On skilled in the art will understand that the distribution of macrochannel diameters can be a normal distribution of diameters or a non-normal distribution diameters.

Imaging

Various aspects of the application provide for imaging of a biological hard tissue so as to provide an anatomic external shape for the matrix. Imaging of a hard tissue can be according to a variety of means conventional in the art. Imaging can be according to, for example, X-ray, computed tomography (CT), microcomputed tomography (μCT), magnetic resonance imaging (MRI), and/or ultrasound.

In some embodiments, imaging of a tissue produces a three-dimensional image (or data representing such) of the structure (see e.g., Example 1; Example 6). The resulting data can be reformatted in various planes (e.g., multiplanar reformatted imaging) or, preferably, as a volumetric representation of the structure. As known in the art, a software program can be used to to build a volume by “stacking” individual image slices one on top of the other, including orthogonal plan, oblique plane, and curved plane reconstruction. Methods of image reconstruction include, but are not limited to multiplanar reconstruction, maximum-intensity projection, and minimum-intensity projection.

One skilled in the art can select suitable threshold values of radiodensity corresponding to the target tissue. Threshold levels can be set using edge detection image processing algorithms. From this, a 3-dimensional model can be constructed and displayed. Multiple models can be constructed from various different thresholds, allowing different representations of differing anatomical components such as bone, muscle, and cartilage. Where different structures have similar radiodensity, segmentation can remove unwanted structures from the image.

In addition to the exterior anatomic contour of the hard tissue, various internal structures and features can be imaged. Imaging of hard tissue internal structures is within the skill of the art. As an example, internal bone trabeculae structures of the hard tissue of interest can be imaged.

The resultant 3-dimensional image can be used to fabricate a matrix scaffold having the same external and internal anatomic shape and features as the hard tisse of interest (see e.g., Example 1; Example 6).

Fabrication

In various aspects of the application, biocompatible matrix materials are fabricated into an artificial structure (i.e., scaffold) capable of supporting three-dimensional tissue formation having similar shape and/or function as a hard tissue of interest.

Fabrication of biocompatible matrix materials into a shaped 3-dimensional scaffold can be according to a variety of methods known to the art (see e.g., Example 1; Example 6). Scaffold synthesis techniques include, but are not limited to, nanofiber self-assembly (e.g., hydrogel scaffolds), textile technologies (e.g., non-woven polyglycolide structures), solvent casting and particulate leaching, gas foaming, emulsification/freeze-drying, thermally induced phase separation, CAD/CAM technologies, or a combination of these techniques. Preferably, biocompatible matrix materials are fabricated into a shaped 3-dimensional scaffold via computer aided design/manufacturing (CAD/CAM) technologies.

In CAD/CAM technologies of scaffold fabrication, first a three-dimensional structure is designed using computer aided design (CAD) software and then the scaffold is generated by computer aided manufacture (CAM) process. CAM processes for scaffold fabrication include, for example, using ink jet printing of polymer powders (e.g., Bioplotter, Envisiontec, Gladbeck, Germany) or through rapid prototyping technology such as fused deposition modeling (FDM). Scaffold fabrication using a bioplotter, or similar device, provides the advantage of co-deposition of live cells (e.g., progenitor cells). For example, multiple printing/deposition heads can be used in the fabrication of materials, co-deposition of cells, and/or addition of agents such as growth factors and the like so as to provide for a fabricated scaffold with internal porosity features and seeded progenitor cells and/or additional agents within the scaffold material and/or its pores/channels.

Scaffold fabrication via CAD/CAM technologies usually employs 3-dimensional data of the target hard tissue. As described above, the image data can be obtained from a subject's own tissue or from similar tissue from other than the subject. Software can import 3-dimensional volume data and generate a plotting pathway for deposition of the matrix material. For example, dxf-data can be prepared by processing CT scanned images or obtained from medical CAD programs like VOX1M or MIMICS, which reconstructs a 3D model from D1COM images. The 3-dimensional model can be an integral solid of which body surrounded by surface objects. Once a 3-dimensional volume data file (e.g., a dxf file) is constructed, the size, alignment, and position is adjusted per the dispensing layouts and channel configurations. Such adjustment is within the ordinary skill in the art. In a typical procedure, a selected matrix polymer material (e.g., PCL) is placed inside the container of the dispensing module, and the module heated to a pre-optimized temperature to keep the polymer melted with appropriate viscosity for dispensing. The polymer solution can also be prepared using a solvent. With solvent, the desired viscosity can be controlled by concentration of solute, and in some embodiments, no heat is required. The polymer solution can be dispensed in air or in liquid, optionally with chemicals required for solidification. For example, melted PCL can be dispensed in air.

For CAM fabrication techniques, the pore size of the resulting scaffold can be determined by distance between strands. The strand size can be determined by, for example, viscosity of solution, needle inner diameter, and dispensing speed. Preferably, pore size parameters are determined prior to fabrication of a 3-dimensional structure, as is within the skill of the art.

Delivery of Cells

In various embodiments of the modules of the application, progenitor cells are introduced (e.g., implanted, injected, infused, or seeded) into or onto an artificial structure (e.g., a scaffold comprising a matrix material) capable of supporting three-dimensional tissue formation. The tissue progenitor cells can be co-introduced or sequentially introduced. Where differing types of progenitor cells are employed (e.g., bone progenitor cells and cartilage progenitor cells), they can be introduced in the same spatial position, similar spatial positions, or different spatial positions, relative to each other. Preferably, bone progenitor cells and cartilage progenitor cells are introduced into or onto different areas of the matrix material, and more preferably introduced into different layers of the matrix so as to mimic an internal bone layer and an external cartilage layer characteristic of a joint. It is contemplated that more than one types of bone progenitor cells can be introduced into the matrix. Similarly, it is contemplated that more than one type of cartilage progenitor cell can be introduced into the matrix.

Progenitor cells can be introduced into the matrix material by a variety of means known to the art (see e.g., Example 1; Example 4). Methods for the introduction (e.g., infusion, seeding, injection, etc.) of progenitor cells into or into the matrix material are discussed in, for example, Ma and Elisseeff, ed. (2005) Scaffolding In Tissue Engineering, CRC, ISBN 1574445219; Saltzman (2004) Tissue Engineering: Engineering Principles for the Design of Replacement Organs and Tissues, Oxford ISBN 019514130X; Minuth et al. (2005) Tissue Engineering: From Cell Biology to Artificial Organs, John Wiley & Sons, ISBN 3527311866. For example, progenitor cells can be introduced into or onto the matrix by methods including hydrating freeze-dried scaffolds with a cell suspension (e.g., at a concentration of 100 cells/ml to several million cells/ml). Methods of addition of additional agents vary, as discussed below.

Preferably, progenitor cells are introduced into the matrix at the time of fabrication. For example, progenitor cells can be introduced into the scaffold by a bioplotter, or other similar device, during or near the time when biocompatible polymer layers are formed into a 3-dimensional scaffold (e.g., cell printing).

Methods of culturing and differentiating progenitor cells in or on scaffolds are generally known in the art (see e.g., Saltzman (2004) Tissue Engineering: Engineering Principles for the Design of Replacement Organs and Tissues, Oxford ISBN 019514130X; Vunjak-Novakovic and Freshney, eds. (2006) Culture of Cells for Tissue Engineering, Wiley-Liss, ISBN 0471629359; Minuth et al. (2005) Tissue Engineering: From Cell Biology to Artificial Organs, John Wiley & Sons, ISBN 3527311866). As will be appreciated by one skilled in the art, the time between progenitor cell introduction into or onto the matrix and engrafting the resulting matrix can vary according to particular application. Incubation (and subsequent replication and/or differentiation) of the engineered composition containing bone progenitor cells and/or cartilage progenitor cells in or on the matrix material can be, for example, at least in part in vitro, substantially in vitro, at least in part in vivo, or substantially in vivo. Determination of optimal culture time is within the skill of the art. A suitable medium can be used for in vitro progenitor cell infusion, differentiation, or cell transdifferentiation (see e.g., Vunjak-Novakovic and Freshney, eds. (2006) Culture of Cells for Tissue Engineering, Wiley-Liss, ISBN 0471629359; Minuth et al. (2005) Tissue Engineering: From Cell Biology to Artificial Organs, John Wiley & Sons, ISBN 3527311866). The culture time can vary from about an hour, several hours, a day, several days, a week, or several weeks. The quantity and type of cells present in the matrix can be characterized by, for example, morphology by ELISA, by protein assays, by genetic assays, by mechanical analysis, by RT-PCR, and/or by immunostaining to screen for cell-type-specific markers (see e.g., Minuth et al. (2005) Tissue Engineering: From Cell Biology to Artificial Organs, John Wiley & Sons, ISBN 3527311866).

For tissue modules using small scaffolds (<100 cubic millimeters in size), in vitro medium can be changed manually, and additional agents added periodically (e.g., every 3-4 days). For larger scaffolds, the culture can be maintained, for example, in a bioreactor system, which may use a minipump for medium change. The minipump can be housed in an incubator, with fresh medium pumped to the matrix material of the scaffold. The medium circulated back to, and through, the matrix can have about 1% to about 100% fresh medium. The pump rate can be adjusted for optimal distribution of medium and/or additional agents included in the medium. The medium delivery system can be tailored to the type of tissue or organ being manufactured. All culturing is preferably performed under sterile conditions.

The present teachings include methods for optimizing the density of progenitor cells (e.g., bone progenitor cells and cartilage progenitor cells) (and their lineage derivatives) so as to maximize the regenerative outcome of a hard tissue module. Cell densities in a matrix can be monitored over time and at end-points. Tissue properties can be determined, for example, using standard techniques known to skilled artisans, such as histology, structural analysis, immunohistochemistry, biochemical analysis, and mechanical properties. As will be recognized by one skilled in the art, the cell densities of progenitor cells can vary according to, for example, progenitor type, tissue or organ type, matrix material, matrix volume, infusion method, seeding pattern, culture medium, growth factors, incubation time, incubation conditions, and the like. Generally, for both bone progenitor cells and cartilage progenitor cells, the cell density of each cell type in a matrix can be, independently, from 0.0001 million cells (M) ml⁻¹ to about 1000 M ml⁻¹. For example, the tissue progenitor cells and the vascular progenitor cells can each be present in the matrix at a density of about 0.001 M ml⁻¹, 0.01 M ml⁻¹, 0.1 M ml⁻¹, 1 M ml⁻¹, 5 M ml⁻¹, 10 M ml⁻¹, 15 M ml⁻¹, 20 M ml⁻¹, 25 M ml⁻¹, 30 M ml⁻¹, 35 M ml⁻¹, 40 M ml⁻¹, 45 M ml⁻¹, 50 M ml⁻¹, 55 M ml⁻¹, 60 M ml⁻¹, 65 M ml⁻¹, 70 M ml⁻¹, 75 M ml⁻¹, 80 M ml⁻¹, 85 M ml⁻¹, 90 M ml⁻¹, 95 M ml⁻¹, 100 M ml⁻¹, 200 M ml⁻¹, 300 M ml⁻¹, 400 M ml⁻¹, 500 M ml⁻¹, 600 M ml⁻¹, 700 M ml⁻¹, 800 M ml⁻¹, or 900 M ml⁻¹.

In some embodiments, a tissue module can comprise progenitor cells at a density of about 0.0001 million cells (M) ml⁻¹ to about 1000 M ml⁻¹. In some configurations, a tissue module can comprise progenitor cells at a density of at least about 1 M ml⁻¹ up to about 100 M ml⁻¹. In some configurations, a tissue module can comprise progenitor cells at a density of at least about 5 M ml⁻¹ up to about 95 M ml⁻¹. In some configurations, a tissue module can comprise progenitor cells at a density of at least about 10 M ml⁻¹ up to about 90 M ml⁻¹. In some configurations, a tissue module can comprise progenitor cells at a density of at least about 15 M ml⁻¹ up to about 85 M ml⁻¹. In some configurations, a tissue module can comprise progenitor cells at a density of at least about 20 M ml⁻¹ up to about 80 M ml⁻¹. In some configurations, a tissue module can comprise progenitor cells at a density of at least about 25 M ml⁻¹ up to about 75 M ml⁻¹. In some configurations, a tissue module can comprise progenitor cells at a density of at least about 30 M ml⁻¹ up to about 70 M ml⁻¹. In some configurations, a tissue module can comprise progenitor cells at a density of at least about 35 M ml⁻¹ up to about 65 M ml⁻¹. In some configurations, a tissue module can comprise progenitor cells at a density of at least about 40 M ml⁻¹ up to about 60 M ml⁻¹. In some configurations, a tissue module can comprise progenitor cells at a density of at least about 45 M ml⁻¹ up to about 55 M ml⁻¹. In some configurations, a tissue module can comprise progenitor cells at a density of at least about 45 M ml⁻¹ up to about 50 M ml⁻¹. In some configurations, a tissue module can comprise progenitor cells at a density of at least about 50 M ml⁻¹ up to about 55 M ml⁻¹.

Bone progenitor cells and cartilage progenitor cells can be introduced at various ratios in or on the matrix. As will be recognized by one skilled in the art, the cell ratio of bone progenitor cells to cartilage progenitor cells can vary according to, for example, type of progenitor cells, target tissue type, matrix material, matrix volume, infusion method, seeding pattern, culture medium, growth factors, incubation time, and/or incubation conditions. In some embodiments, the ratio of bone progenitor cells to cartilage progenitor cells can be about 100:1 to about 1:100. For example, the ratio of bone progenitor cells to cartilage progenitor cells can be about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, or 1:20.

In some configurations, the ratio of bone progenitor cells to cartilage progenitor cells can be from about 20:1 up to about 1:20. In some configurations, the ratio of bone progenitor cells to cartilage progenitor cells can be from about 19:1 to about 1:19. In some configurations, the ratio of bone progenitor cells to cartilage progenitor cells can be from about 18:1 to about 1:18. In some configurations, the ratio of bone progenitor cells to cartilage progenitor cells can be from about In some configurations, the ratio of bone progenitor cells to cartilage progenitor cells can be from about 17:1 to about 1:17. In some configurations, the ratio of bone progenitor cells to cartilage progenitor cells can be from about In some configurations, the ratio of bone progenitor cells to cartilage progenitor cells can be from about 16:1 to about 1:16. In some configurations, the ratio of bone progenitor cells to cartilage progenitor cells can be from about In some configurations, the ratio of bone progenitor cells to cartilage progenitor cells can be from about 15:1 to about 1:15. In some configurations, the ratio of bone progenitor cells to cartilage progenitor cells can be from about 14:1 to about 1:14. In some configurations, the ratio of bone progenitor cells to cartilage progenitor cells can be from about 13:1 to about 1:13. In some configurations, the ratio of bone progenitor cells to cartilage progenitor cells can be from about 12:1 to about 1:12. In some configurations, the ratio of bone progenitor cells to cartilage progenitor cells can be from about 11:1 to about 1:11. In some configurations, the ratio of bone progenitor cells to cartilage progenitor cells can be from about 10:1 to about 1:10. In some configurations, the ratio of bone progenitor cells to cartilage progenitor cells can be from about 9:1 to about 1:9. In some configurations, the ratio of bone progenitor cells to cartilage progenitor cells can be from about In some configurations, the ratio of bone progenitor cells to cartilage progenitor cells can be from about 8:1 to about 1:8. In some configurations, the ratio of bone progenitor cells to cartilage progenitor cells can be from about 7:1 to about 1:7. In some configurations, the ratio of bone progenitor cells to cartilage progenitor cells can be from about 6:1 to about 1:6. In some configurations, the ratio of bone progenitor cells to cartilage progenitor cells can be from about 5:1 to about 1:5. In some configurations, the ratio of bone progenitor cells to cartilage progenitor cells can be from about 4:1 to about 1:4. In some configurations, the ratio of bone progenitor cells to cartilage progenitor cells can be from about In some configurations, the ratio of bone progenitor cells to cartilage progenitor cells can be from about 3:1 to about 1:3. In some configurations, the ratio of bone progenitor cells to cartilage progenitor cells can be from about In some configurations, the ratio of bone progenitor cells to cartilage progenitor cells can be from about 2:1 to about 1:2.

In some embodiments, one or more cell types in addition to a first type of bone progenitor cells and a first type of cartilage progenitor cells can be introduced into or onto the matrix material. Such additional cell type can be selected from those discussed above, and/or can include (but not limited to) skin cells, liver cells, heart cells, kidney cells, pancreatic cells, lung cells, bladder cells, stomach cells, intestinal cells, cells of the urogenital tract, breast cells, skeletal muscle cells, skin cells, bone cells, cartilage cells, keratinocytes, hepatocytes, gastro-intestinal cells, epithelial cells, endothelial cells, mammary cells, skeletal muscle cells, smooth muscle cells, parenchymal cells, osteoclasts, or chondrocytes. These cell-types can be introduced prior to, during, or after introduction of the first type of bone progenitor cells and/or the first type of cartilage progenitor cells. Such introduction may take place in vitro or in vivo. When the cells are introduced in vivo, the introduction may be at the site of the tissue module or at a site removed therefrom. Exemplary routes of administration of the cells include injection and surgical implantation.

Method of Treatment

Various embodiments of the tissue modules of the application hold significant clinical value because of their biomaterials, anatomic shape, internal structural features, and/or multilayer and/or composite composition which more precisely mimics target hard tissue as compared to other engineered tissues produced by other means known to the art. It is these features, at least in part, which sets the tissue modules disclosed herein apart from other conventional treatment options.

Another provided aspect is a method of treating a tissue defect in a subject by implanting a tissue module described herein into a subject in need thereof. A determination of the need for treatment will typically be assessed by a history and physical exam consistent with the tissue defect at issue. Subjects with an identified need of therapy include those with a diagnosed tissue defect. The subject is preferably an animal, including, but not limited to, mammals, reptiles, and avians, more preferably horses, cows, dogs, cats, sheep, pigs, and chickens, and most preferably human.

As an example, a subject in need may have damage to a tissue, and the method provides an increase in biological function of the tissue by at least 5%, 10%, 25%, 50%, 75%, 90%, 100%, or 200%, or even by as much as 300%, 400%, or 500%. As yet another example, the subject in need may have a disease, disorder, or condition, and the method provides an engineered tissue module sufficient to ameliorate or stabilize the disease, disorder, or condition. For example, the subject may have a disease, disorder, or condition that results in the loss, atrophy, dysfunction, or death of cells. Exemplary treated conditions include arthritis; osteoarthritis; osteoporosis; osteochondrosis; osteochondritis; osteogenesis imperfecta; osteomyelitis; osteophytes (i.e., bone spurs); achondroplasia; costochondritis; chondroma; chondrosarcoma; herniated disk; Klippel-Feil syndrome; osteitis deformans; osteitis fibrosa cystica, a congenital defect that results in the absence of a tissue; accidental tissue defect or damage such as fracture, wound, or joint trauma; an autoimmune disorder; diabetes (e.g., Charcot foot); cancer; a disease, disorder, or condition that requires the removal of a tissue (e.g., tumor resection); and/or a disease, disorder, or condition that affects the trabecular to cortical bone ratio. For example, a tissue module described herein can be implanted in a subject who would otherwise need to undergo an osteochondral autograft. In a further example, the subject in need may have an increased risk of developing a disease, disorder, or condition that is delayed or prevented by the method.

Implantation of a hard tissue module described herein is within the skill of the art. The matrix and/or cellular assembly can be either fully or partially implanted into a tissue or organ of the subject to become a functioning part thereof. Preferably, the implant initially attaches to and communicates with the host through a cellular monolayer. In some embodiments, over time, the introduced cells can expand and migrate out of the polymeric matrix to the surrounding tissue. After implantation, cells surrounding the tissue module can enter through cell migration. The cells surrounding the tissue module can be attracted by biologically active materials, including biological response modifiers, such as polysaccharides, proteins, peptides, genes, antigens, and antibodies which can be selectively incorporated into the matrix to provide the needed selectivity, for example, to tether the cell receptors to the matrix or stimulate cell migration into the matrix, or both. Generally, the matrix is porous, having interconnecting microchannels and/or macrochannels that allow for cell migration, augmented by both biological and physical-chemical gradients. For example, cells surrounding the implanted matrix can be attracted by biologically active materials including one ore more of VEGF, fibroblast growth factor, transforming growth factor-beta, endothelial cell growth factor, P-selectin, and intercellular adhesion molecule. One of skill in the art will recognize and know how to use other biologically active materials that are appropriate for attracting cells to the matrix.

The methods, compositions, and devices of the application can include concurrent or sequential treatment with one or more of enzymes, ions, growth factors, and biologic agents, such as thrombin and calcium, or combinations thereof. The methods, compositions, and devices of the application can include concurrent or sequential treatment with non-biologic and/or biologic drugs.

Added Drugs and/or Diagnostics

In some embodiments, the methods and compositions of the application further comprise additional agents introduced into or onto the matrix. Various agents that can be introduced include, but are not limited to, bioactive molecules, biologic drugs, diagnostic agents, and strengthening agents.

The matrix can further comprise at least one bioactive molecule. In some embodiments, cells of the matrix can be, for example, genetically engineered to express the bioactive molecule or the bioactive molecule can be added to the matrix. The matrix can also be cultured in the presence of the bioactive molecule. The bioactive molecule can be added prior to, during, or after progenitor cells (when present) are introduced to the matrix. Preferably, the matrix includes at least one osteoinductive and/or chondroinductive cytokine

Non-limiting examples of bioactive molecules include activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin, fibronectin receptor α₅β₁, Factor X, HB-EGF, HBNF, HGF, HUAF, heart derived inhibitor of vascular cell proliferation, IFN-gamma, IL1, IGF-2 IFN-gamma, integrin receptors (e.g., various combinations of α subunits (e.g., α₁, α₂, α₃, α₄, α₅, α₆, α₇, α₈, α₉, α_{E}, α_{V}, α_{IIb}, α_{L}, α_{M}, α_{X}) andβ subunits (e.g., β₁, β₂, β₃, β₄, β₅, β₆, β₇, and β₈)), K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPARγ ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-β, Tie 1, Tie2, TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF₁₆₄, VEGI, EG-VEGF, VEGF receptors, PF4, 16 kDa fragment of prolactin, prostaglandins E1 and E2, steroids, heparin, 1-butyryl glycerol (monobutyrin), and nicotinic amide. In other preferred embodiments, the matrix includes a chemotherapeutic agent or immunomodulatory molecule. Such agents and molecules are known to the skilled artisan. Preferably, the matrix includes a TGFβ, bFGF, VEGF, or PDGF, or some combination thereof. More preferably, the matrix includes at least TGFβ3. As shown herein, cytokine TGFβ3, infused into microchanneled scaffolds can enhance articular cartilage regeneration (See e.g., Example 7, Example 8).

Biologic drugs that can be added to the compositions of the application include immunomodulators and other biological response modifiers. A biological response modifier generally encompasses a biomolecule (e.g., peptide, peptide fragment, polysaccharide, lipid, antibody) that is involved in modifying a biological response, such as the immune response or tissue growth and repair, in a manner which enhances a particular desired therapeutic effect, for example, the cytolysis of bacterial cells or the growth of tissue-specific cells or vascularization. Biologic drugs can also be incorporated directly into the matrix component. Those of skill in the art will know, or can readily ascertain, other substances which can act as suitable non-biologic and biologic drugs.

Biomolecules can be incorporated into the matrix, causing the biomolecules to be imbedded within. Alternatively, chemical modification methods may be used to covalently link a biomolecule on the surface of the matrix. The surface functional groups of the matrix components can be coupled with reactive functional groups of the biomolecules to form covalent bonds using coupling agents well known in the art such as aldehyde compounds, carbodiimides, and the like. Additionally, a spacer molecule can be used to gap the surface reactive groups and the reactive groups of the biomolecules to allow more flexibility of such molecules on the surface of the matrix. Other similar methods of attaching biomolecules to the interior or exterior of a matrix will be known to one of skill in the art.

Compositions of the application can also be modified to incorporate a diagnostic agent, such as a radiopaque agent. The presence of such agents can allow the physician to monitor the progression of healing and/or growth occurring internally. Such compounds include barium sulfate as well as various organic compounds containing iodine. Examples of these latter compounds include iocetamic acid, iodipamide, iodoxamate meglumine, iopanoic acid, as well as diatrizoate derivatives, such as diatrizoate sodium. Other contrast agents which can be utilized in the compositions of the application can be readily ascertained by those of skill in the art and may include the use of radiolabeled fatty acids or analogs thereof.

The concentration of agent in the composition will vary with the nature of the compound, its physiological role, and desired therapeutic or diagnostic effect. A therapeutically effective amount is generally a sufficient concentration of therapeutic agent to display the desired effect without undue toxicity. A diagnostically effective amount is generally a concentration of diagnostic agent which is effective in allowing the monitoring of the integration of the construct, while minimizing potential toxicity. In any event, the desired concentration in a particular instance for a particular compound is readily ascertainable by one of skill in the art.

The matrix composition can be enhanced, or strengthened, through the use of such supplements as human serum albumin (HSA), hydroxyethyl starch, dextran, or combinations thereof. The solubility of the matrix compositions can also be enhanced by the addition of a nondenaturing nonionic detergent, such as polysorbate 80. Suitable concentrations of these compounds for use in the compositions of the application will be known to those of skill in the art, or can be readily ascertained without undue experimentation. The matrix compositions can also be further enhanced by the use of optional stabilizers or diluent. The proper use of these would be known to one of skill in the art, or can be readily ascertained without undue experimentation.

Agents can be introduced into or onto the matrix via a carrier based system, such as an encapsulation vehicle. For example, growth factors can be micro-encapsulated to provide for enhanced stability and/or prolonged delivery. Encapsulation vehicles include, but are not limited to, microparticles, liposomes, microspheres, or the like, or a combination of any of the above to provide the desired release profile in varying proportions. Other methods of controlled-release delivery of agents will be known to the skilled artisan. Moreover, these and other systems can be combined and/or modified to optimize the integration/release of agents within the matrix.

Carrier based systems for incorporation of various agents into or onto the matrix can: provide for enhanced intracellular delivery; tailor biomolecule/agent release rates; increase and/or accelerate functional integration of layers; increase the proportion of agent that reaches its site of action; improve the transport of the agent to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; and/or improve shelf life of the product.

Polymeric microspheres can be produced using naturally occurring or synthetic polymers and are particulate systems in the size range of 0.1 to 500 μm. Polymeric micelles and polymeromes are polymeric delivery vehicles with similar characteristics to microspheres and can also facilitate encapsulation and matrix integration of the agents described herein. Fabrication, encapsulation, and stabilization of microspheres for a variety of payloads are within the skill of the art (see e.g., Varde & Pack (2004) Expert Opin. Biol. 4(1) 35-51). Release rate of microspheres can be tailored by type of polymer, polymer molecular weight, copolymer composition, excipients added to the microsphere formulation, and microsphere size. Polymer materials useful for forming microspheres include PLA, PLGA, PLGA coated with DPPC, DPPC, DSPC, EVAc, gelatin, albumin, chitosan, dextran, DL-PLG, SDLMs, PEG (e.g., ProMaxx), sodium hyaluronate, diketopiperazine derivatives (e.g., Technosphere), calcium phosphate-PEG particles, and/or oligosaccharide derivative DPPG (e.g., Solidose). Encapsulation can be accomplished, for example, using a water/oil single emulsion method, a water-oil-water double emulsion method, or lyophilization. Several commercial encapsulation technologies are available (e.g., ProLease®, Alkerme).

Polymeric hydrogels can be used to integrate various agents into the matrix. For example, a polymeric hydrogel including one or more agents can be form a layer, or a part of a layer, of a composite tissue module as described herein. As another example, a polymeric hydrogel including one or more agents can be introduced into pores, microchannels, and/or macrochannels of the matrix.

“Smart” polymeric carriers can be used to integrate agents with the matrix (see generally, Stayton et al. (2005) Orthod Craniofacial Res 8, 219-225; Wu et al. (2005) Nature Biotech (2005) 23(9), 1137-1146). Carriers of this type utilize polymers that are hydrophilic and stealth-like at physiological pH, but become hydrophobic and membrane-destabilizing after uptake into the endosomal compartment (i.e., acidic stimuli from endosomal pH gradient) where they enhance the release of the cargo molecule into the cytoplasm. Design of the smart polymeric carrier can incorporate pH-sensing functionalities, hydrophobic membrane-destabilizing groups, versatile conjugation and/or complexation elements to allow the drug incorporation, and an optional cell targeting component. Polymeric carriers include, for example, the family of poly(alkylacrylic acid) polymers, specific examples including poly(methylacrylic acid), poly(ethylacrylic acid) (PEAA), poly(propylacrylic acid) (PPAA), and poly(butylacrylic acid) (PBAA), where the alkyl group is progressively increased by one methylene group. Various linker chemistries are available to provide degradable conjugation sites for proteins, nucleic acids, and/or targeting moieties. For example, pyridyl disulfide acrylate (PDSA) monomer allow efficient conjugation reactions through disulfide linkages that can be reduced in the cytoplasm after endosomal translocation of the agent(s).

Liposomes can be used to integrate gents with the matrix. The agent carrying capacity and release rate of liposomes can depend on the lipid composition, size, charge, drug/lipid ratio, and method of delivery. Conventional liposomes are composed of neutral or anionic lipids (natural or synthetic). Commonly used lipids are lecithins such as (phosphatidylcholines), phosphatidylethanolamines (PE), sphingomyelins, phosphatidylserines, phosphatidylglycerols (PG), and phosphatidylinositols (PI). Liposome encapsulation methods are commonly known in the arts (Galovic et al. (2002) Eur. J. Pharm. Sci. 15, 441-448; Wagner et al. (2002) J. Liposome Res. 12, 259-270). Targeted liposomes and reactive liposomes can also be used in combination with the agents and matrix. Targeted liposomes have targeting ligands, such as monoclonal antibodies or lectins, attached to their surface, allowing interaction with specific receptors and/or cell types. Reactive or polymorphic liposomes include a wide range of liposomes, the common property of which is their tendency to change their phase and structure upon a particular interaction (eg, pH-sensitive liposomes) (see e.g., Lasic (1997) Liposomes in Gene Delivery, CRC Press, Fla.).

Toxicity and therapeutic efficacy of agents discussed herein can be determined by standard pharmaceutical procedures in cell cultures and/or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where large therapeutic indices are preferred.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### References Cited

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Design and Fabrication of Human-Shaped Synovial Joint Condyles from Skeletal Images

The following example demonstrates generation of an engineered human-shaped proximal tibial condyle of the knee joint from polycaprolactone (PCL).

First, the joint is imaged. Imaging can be according to, for example, X ray, CT, microcomputed tomography (pCT), magnetic resonance imaging (MRI), and/or ultrasound. The imaged joint can be, for example, a synovial joint such as the hip joint, the knee joint, the elbow joint, the phalanges, or the temporomandibular joint. For this example, the proximal tibial condyle of the knee joint was imaged.

Second, multiple image slices of the synovial joint were reconstructed into 3D structures incorporating not only the external anatomic contour but also internal bone trabeculae structures via computer aided design (CAD). The result of this step was a “.dxf” file of AutoCAD containing the 3D volume data.

Third, computer aided manufacturing (CAM) was used with a Bioplotter (Envisiontec, Gladbeck, Germany) or a rapid prototyping device to fabricate 3D biocompatible scaffolds from a variety of biocompatible polymers using 3D Dispenser. Live cells were deposited using Bioplotter in biocompatible materials. Live cells were not deposited with the rapid prototyping device. A variety of biocompatible materials were used, such as polycaprolactone (PCL) and poly(ethylene) oxide. PCL is a biodegradable polymeric material that simulates the mechanical properties of bone.

A Bioplotter was used to fabricate 3D scaffolds in the shape of human proximal tibial condyle. The following provides an overview that can be adapted for the fabrication step. Eembedded software imports 3D volume data from the “.dxf” file (AutoCAD) and generates the plotting pathway of the nozzle. The dxf-data can be prepared by processing CT scanned images or obtained from medical CAD programs like VOX1M or MIMICS, which reconstructs a 3D model from D1COM images. The 3D model should be an integral solid of which body surrounded by surface objects. Once a 3D dxf file is constructed, the size, alignment, and position is adjusted per the dispensing layouts and channel configurations. The selected polymer material (e.g., PCL) is placed inside the container of the dispensing module. The module is heated by the pre-optimized temperature to keep the polymer melted with appropriate viscosity for dispensing. The polymer solution can also be prepared using solvent. With solvent, the desired viscosity is controlled by concentration of solute and no heat is generally applied. Then, the polymer solution is dispensed in air or in liquid with chemicals required for solidification. The melted PCL is generally dispensed in air. The pore size is determined by distance between strands. The strand size is determined by viscosity of solution, needle inner diameter, and dispensing speed. For precise control of pore size, these parameters are determined prior to fabrication of a whole 3D structure.

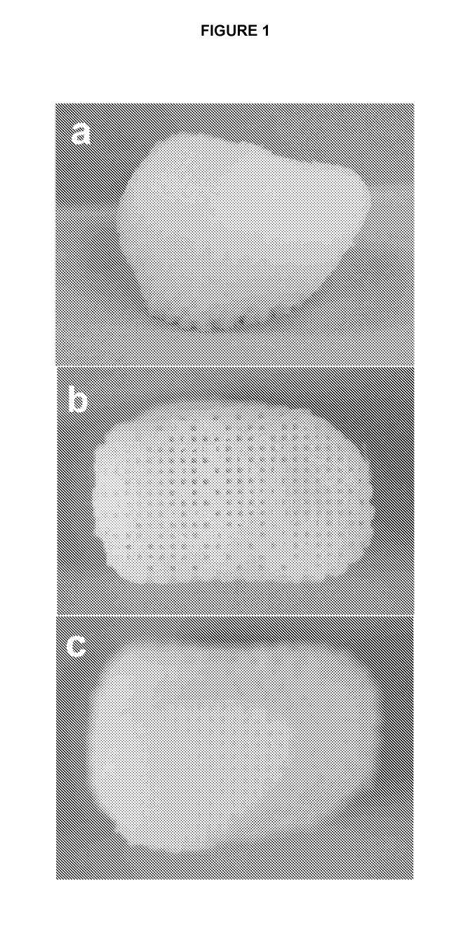

According to the above overview, a 3D scaffold in the shape of human proximal tibial condyle was fabricated having pores and channels with a diameter of 400 um. An exemplary bioplotter-fabricated 3D scaffold in the shape of the proximal tibial condyle of the human knee joint is shown in FIG. 1. Each of these structures have internal pores, porosity, and inter-pore connections that can be fine-tuned for optimization of the in vivo regeneration outcome. The size of pores and channels can be fine-tuned from the millimeter range to micrometer range to accommodate tissue regeneration needs. Pores and channels can be used for seeding cells and/or growth factors, or serve as conduits for vascularization as well as perfusion needs.

Fourth, the bioplotted porous scaffolds with microchannels and inter-porosity were anchored to a hydrogel such as poly(ethylene glycol) (PEG) hydrogel by sequential polymerization of the PEG.

### Example 2

### Design and Fabrication of Human-Shaped Femoral Condyle of the Hip Joint

A human shaped femoral condyle of the hip joint engineered from polycaprolactone (PCL) was formed in accordance with the methods described in Example 1. Pores and channels of the engineered joint had a diameter of 400 um. Cells and/or growth factors are deposited in the PCL. An exemplary bioplotter-fabricated 3D scaffold in the shape of the femoral condyle of the hip joint is shown in FIG. 2.

In addition, a cortical shell was fabricated to simulate cortical bone (see e.g., FIG. 2). Similar to the structure exemplified in FIG. 1, internal pores and channels simulate bone trabeculae (see e.g., FIG. 2).

### Example 3

### Design and Fabrication of Human-Shaped Mandibular Condyle of the Temporomandibular Join

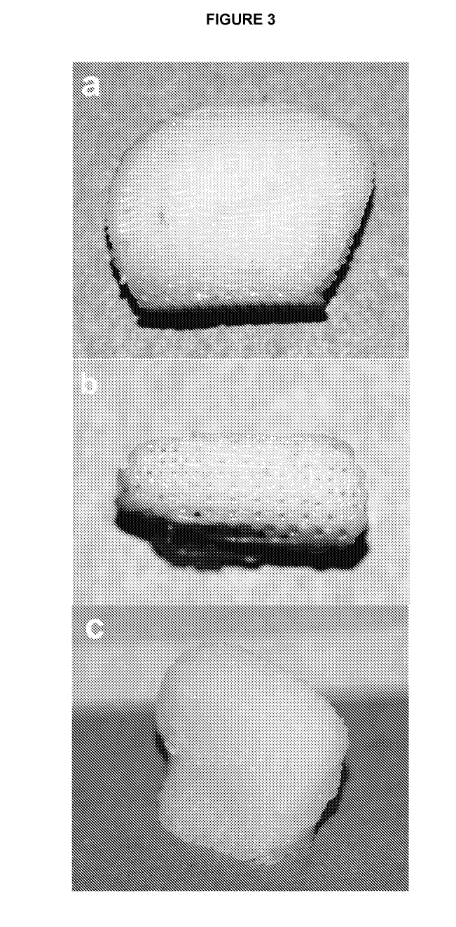

A human-shaped mandibular condyle of the human temporomandibular joint engineered from polycaprolactone (PCL) was formed in accordance with the methods described in Example 1. Pores and channels of the engineered joint had a diameter of 400 um. Cells and/or growth factors were deposited in the PCL. An exemplary bioplotter-fabricated 3D scaffold in the shape of the mandibular condyle of the human temporomandibular joint is shown in FIG. 3. Similar to the structure exemplified in FIG. 1, internal pores and channels simulate bone trabeculae (see e.g., FIG. 3).

### Example 4

### Design and Fabrication of Composite Joint

The following example details design and fabrication of a human-shaped proximal tibia condyle of the knee joint engineered from two composite materials, a hydrogel material that is anchored to a stiff polymeric material. Hydrogel material simulates articular cartilage, whereas stiff material stimulates subchondral bone.

Human mesenchymal stem cells (hMSCs) from several subjects were expanded on 500 cm² tissue culture plates. Approximately 1×10⁶ cells were plated on each plate and within 10 days, the number of viable hMSCs from each plate was about 1×10⁶. For osteogenic differentiation, hMSCs were exposed to DMEM with 100 nM dexamethasone, 60 pg/mL LAscorbic Acid-2-Phosphate (AsAP), 100 mM β-Glycerophosphate. Chondrogenic differentiation was achieved using a three-dimensional encapsulation within a PEG hydrogel. Briefly, expanded hMSCs were rinsed twice with PBS, followed by 1× solution of Trypsin (0.25% Trypsin, 1 mM EDTA) (Atlanta Biologicals, Atlanta, Ga.). Cells were removed and counted using a hemacytometer and centrifuged at 1000 rpm for 10 min. The pellets were resuspended in PEG hydrogel solution and exposed to long-wavelength UV light (365 nm) for 3 min.

Generation of a human-shaped proximal tibia condyle of the knee joint from polycaprolactone (PCL) was formed in accordance with the methods described in Example 1. PCL is a stiff biodegradable polymeric material that simulates the mechanical properties of bone. As described above, the pores and channels can provide for seeding cells and/or growth factors, or serve as conduits for vascularization. Osteoblasts or stem cell-derived osteoblasts were seeded in the PCL. Growth factors were deposited in the PCL.

The PCL scaffold was inverted and immersed in PEG hydrogel solution with a depth of 2 mm. The initial polymerization step increased the viscosity of the PEG hydrogel to limit the amount of PEG to be absorbed within the interconnected pores of the PCL scaffold. The fabricated hydrogel-PCL composite construct was then further exposed to UV light for an additional 12 min. The PCL-PEG hydrogel construct, now containing hMSC-derived chondrocytes within at least the “cartilage” portion (i.e., the PEG hydrogel outer layer) was cultured in a sterile 125 mL capped beaker in 95% DMEM-High Glucose plus 1% 1× ITS+1 solution, 1% penicillin—streptomycin, 100 μg/mL Sodium Pyruvate, 50 μg/mL AsAP, 40 μg/mL L-Proline, 100 nM Dexamethasone, 10 ng/mL TGF-β3. Both osteogenic and chondrogenic differentiation duration was 7 days.

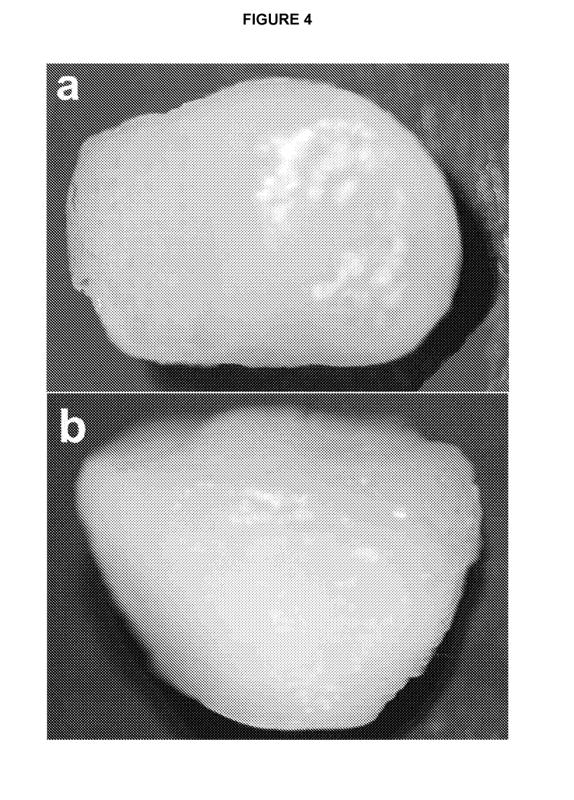

According to the above method, a thin layer (about 1 to 2 mm) of PEG hydrogel, seeded with chondrocytes or stem cell-derived chondrocytes, was anchored to the pores and channels of the PCL scaffold. An exemplary bioplotter-fabricated 3D scaffold (seeded with osteoblasts or stem cell-derived osteoblasts) in the shape of the proximal tibia condyle of the human knee joint coated with a thin PEG hydrogel layer (seeded with chondrocytes or stem cell-derived chondrocytes) is shown in FIG. 4.

### Example 5

### Fabrication and Implantation of Composite Joint

The following example demonstrates in vivo implantation into a rat of a human-shaped proximal tibia condyle of the knee joint engineered from two composite materials.

Fabrication of the 3D scaffold in the shape of the proximal tibia condyle of the human knee joint coated with a thin PEG hydrogel layer was as described in Example 4. As above, MSC derived chondrocytes were seeded in PEG hydrogel, whereas MSC-derived osteoblasts were seeded in PCL.

The engineered joint was implanted into nude rat models. Nude rats are immunodeficient and do not reject human stem cell-seeded implants (see generally, Alhadlaq and Mao, 2003, J Dent Res 82:950-955; Alhadlaq et al., 2004, Ann Biomed Eng 32:911-923). Because the stem cells in this study were from human subject bone marrow samples, the nude rats serve as animal model systems. In a patient model, autologous or allogeneic stem cells could be processed and seeded in joint-shaped scaffolds that are custom-made from the subject's own anatomy.

Immuno-compromised athymic nude rats were sedated and anesthetized with 3% then 1.25-1.5% isoflurane administered gaseous with oxygen. The implantation area was cleansed thrice with alternating applications of betadine solution and 70% ethanol. An incision 50 mm in length was made mid-sagittal linearly across the midsection. Using blunt dissection, pockets were created on each side of the spinal column. Two constructs were implanted per animal, one in each subcutaneous pocket. The incision was closed and secured using 6.0 nylon sutures stitched in a vertical mattress form. An intraperitoneal injection of buprenephrine was given 0.1 mg/kg, postoperatively.

Upon 4 week post operation, CO₂ gas was used to euthanize the rats and the implanted tissue constructs were harvested. Tissue engineered condyles were processed for histology following fixation in 10% Formalin solution. The tissue engineered condyles were embedded in paraffin, sectioned at 5 μm slices, and stained with Hematoxylin and Eosin (H&E).

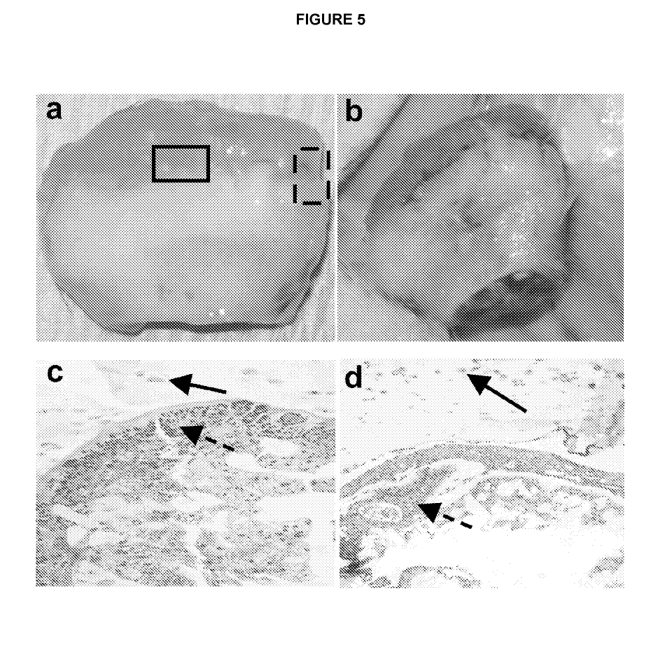

Results showed that the overall anatomical shape of the proximal tibial condyle of the human knee joint was well maintained (see e.g., FIG. 5A). The cartilage layer was distinctive from the underlying bone layer (see e.g., FIG. 5A). Porosity of the PCL was still visible (see e.g., FIG. 5B). The hydrogel-cartilage layer well integrated with surrounding host soft tissue (see e.g., FIG. 5B). Histological examination revealed a cortical like structure with cells that populated both hydrogel-cartilage layer (see e.g., solid arrow of FIG. 5C) and bone-PCL layer (see e.g., dashed arrow of FIG. 5C). This was true with both the center (see e.g., FIG. 5C) and periphery (see e.g., FIG. 5D) of the engineered tibial condyles (see e.g., boxes in FIG. 5A). Areas of vascularization are visible (see e.g., FIG. 5C). Furthermore, blood vessels infiltrated the bone-PCL layer, but stopped on the bone portion of the osteochondral junction without invading into the cartilage-hydrogel layer. At the hydrogel-PCL interface, it was observed that the PEG infiltrated the pores of the PCL.

The above demonstrates engineering of a human-shaped synovial joint condyle from human adult mesenchymal stem cells. Such results support application to subject's in need of total joint replacement. This approach can apply to all joints in the human body including, but not limited to, temporomandibular joint, knee joint, hip joint, elbow joint, should joint, phalangial joints and foot joints, after arthritis and other chronic disorders, congenital anomalies, trauma and tumor resection.

### Example 6

### Bioengineering Design and Fabrication of Anatomically Shaped Synovial Joint

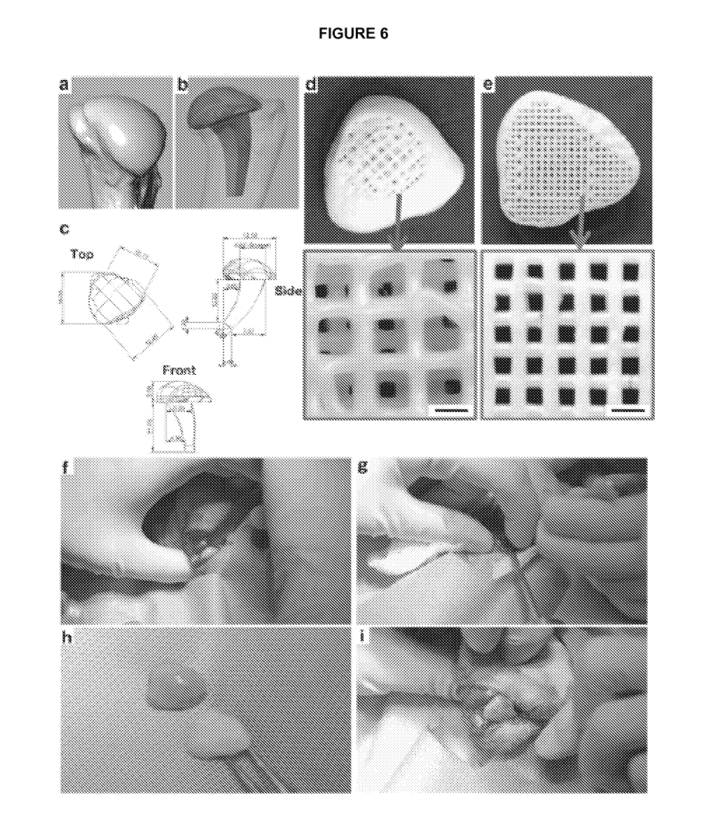

Surface morphology of the proximal humeral condyle of a skeletally matured cadaver rabbit was scanned in 3D (Berding 3D Scanning, Loveland, Ohio) by multi-slice laser scanning at a resolution of 12.7 μm, and manipulated by a conventional CAD software for 3D reconstruction (see e.g., FIG. 6A). The designed scaffold included both articular cartilage and subchondral bone along with an intramedullary stem for surgical fixation (see e.g., FIG. 6B).

A bioengineered graft was designed to replace the entire condylar head of the proximal humerus with a dimension of ˜12×10×5 mm (lengthxwidthxheight) in addition to a tapered ˜11 mm-long stem (see e.g., FIG. 6C). These engineering parameters were used to fabricate a composite polymer scaffold by layer-by-layer deposition using rapid prototyping (Bioplotter™, EnvisionTec, Germany). The composite consisted of 80 wt % polycaprolactone (PCL) (M_{w}˜65,000, Sigma, St. Louis, Mo.) and 20 wt % of hydroxyapatite (HA) (Sigma, St. Louis, Mo.). PCL-HA was then molten in the chamber at 120° C. and dispensed through a 27-gauge metal needle (DL technology, Haverhill, Mass.) and followed the layer path created by 3D morphological data as well as internal microstructures.

The overall dimension of the anatomically shaped scaffold at 12.42×10.11×16.88 mm³ was orders of magnitude greater than the capacity of native nutrient diffusion and waste removal in the range of 100-200 μm. Accordingly, the 200-400 μm microchannels (see e.g., FIG. 6C, D) were designed as conduits for cell homing and vascular supply in vivo. Interconnected microchannels in the size of 200 μm were applied throughout the scaffold except for the top layers down to 1 mm with 400 μm channels (see e.g., FIG. 6C, D inserts). In other words, strand and inter-strand microchannel diameters were 400 μm in the cartilage portion, and 200 μm in the bone portion. The bioengineered graft was sterilized in ethylene oxide for 24 hrs.

### Example 7

### Surgical Joint Replacement by Bioengineered Scaffolds

Anatomically shaped joint constructs are as described in Example 6, except as otherwise noted.

Infusing TGFβ3 in collagen gel into microchannels of PCL-HA scaffold was performed as follows. Transforming growth factor beta 3 (TGFβ3) at a dose of 10 ng/mL (Cell Biosciences, Palo Alto, Calif.) was loaded in 5 mg/mL neutralized bovine type I collagen (Cultrex®, R&D Systems, Minneapolis, Minn.). TGFβ3-loaded collagen solution was then infused into microchannels in the top layer of PCL-HA scaffold and cross-linked for 1 hr in a humidified incubator at 37° C. Of the total 23 rabbits, 10 received bioengineered scaffolds with TGFβ3 infused into the microchannels, whereas the other 13 received bioengineered scaffolds alone without TGFβ3.

A total of 23 skeletally mature, New-Zealand White rabbits received bioengineered scaffolds that surgically replaced the native humeral condyles. Skeletally mature New Zealand white rabbits (3.5-4.0 kg, Harlan, Indianapolis, Ind.) were sedated with ketamine (35 mg/mL) and xylazine (5 mg/mL). Anesthesia was maintained by 1-5% isofluorane inhalation. The right forelimb was prepared for aseptic surgery. A total of 23 rabbits were operated upon: 10 with anatomically shaped scaffolds infused with TGFβ3 into microchannels of the scaffold, and 13 with anatomically shaped scaffolds alone.

TGFβ3 at a concentration of 10 ng/mL was delivered in collagen gel (5 mg/mL) that was infused into the scaffold's microchannels with a surface diameter of 400 μm, followed by crosslinking gelation of collagen gel at 37° C. Purposefully, no stem cells or other cells were transplanted, so to determine whether host-derived cell homing, either in scaffold alone or TGFβ3-delivered scaffolds, was sufficient for tissue regeneration.

With a craniolateral approach to the shoulder joint, the acromial head of the deltoid muscle was tenotomized at its origin and retracted distally. The infraspinatus muscle was tenotomized at its insertion and retracted caudally. The lateral joint capsule was incised from cranial to caudal to expose the humeral head by internal rotation and complete lateral luxation, and then osteotomized. An osteotome and mallet were used to excise the humeral head at its junction with the metaphysis while preserving the greater and lesser tubercles and all soft tissue attachments (see e.g., FIG. 6F), to simulate unipolar joint arthroplasty. A 3.2 mm drill bit on a hand chuck created an intramedullary channel for stem implantation (see e.g., FIG. 6G). Following humeral head excision, the like-shaped, bioengineered scaffold (see e.g., FIG. 6H) was press-fit to replace the excised humeral head (see e.g., FIG. 6I). The joint capsule was closed with a mattress suture, followed by reattachment of the infraspinatus and deltoid tendons. The subcutis was apposed with a continuous suture of 4-0 polydioxanone, followed by closure of skin incision with cruciate sutures of 4-0 nylon. The entire operation per rabbit shoulder joint replacement took approximately 20 minutes. The locomotion of the operated rabbits was video-recorded weekly until euthanized at 8 or 16 wks post-op.

Results showed that within the first approximately 1-4 weeks following joint replacement surgery, the rabbits limped with little use of the operated right forelimb. By approximately 4-6 weeks post surgery, rabbits begun to resume locomotion and weight-bearing with all limbs, including the operated limbs in both scaffold alone and TGFβ3-delivery groups. By approximately 8 weeks post surgery, all rabbits that had received bioengineered joint replacement were able to walk as un-operated, normal rabbits.

### Example 8

### Articular Cartilage Regeneration in Bioengineered Joint

Anatomically shaped joint constructs and surgical joint replacement were as described in Examples 6-7, except as otherwise noted.

Histomorphometric analysis of in vivo bioengineered joints was conducted as follows. The harvested joint samples were embedded in PMMA and sectioned sagittally at 5 μm thickness (HSRL, Jackson, Va.). Randomly selected sections were stained with safranin O, von Kossa, and H&E, respectively. Density and thickness of cartilage-like tissue were calculated (n=10 per group) by image analysis (Leica, Bannockburn, Ill.). Upon confirmation of normal data distribution, Student T test was performed for comparison between with or without delivery of TGFβ3. Glenoid fossa corresponding to the bioengineered condyle was harvested, decalcified, and embedded in paraffin. Medial, central, and lateral sections of glenoid were then prepared and stained with H&E.

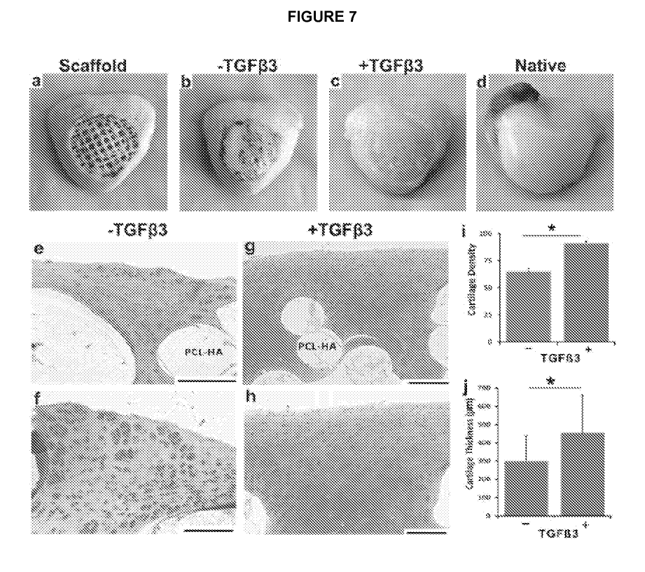

Upon retrieval of in vivo implanted joint replacement constructs at 2 and 4 months post-op (see Example 7), cartilage and subchondral bone regeneration was discovered in bioengineered joint scaffolds. In comparison with un-implanted scaffold sample (see e.g., FIG. 7A), cartilage-like structure was formed on the articular surface in both TGFβ3-free and TGFβ3-delivered of scaffolds per India ink staining (see e.g., FIG. 7B, C, respectively). There was somewhat evenly distributed cartilage-like tissue on the articular surface of TGFβ3-delivered sample (see e.g., FIG. 7C), in comparison with TGFβ3-free samples (see e.g., FIG. 7B). The native articular cartilage surface provides a baseline (see e.g., FIG. 7D). TGFβ3-loaded sample (see e.g., FIG. 7C) showed more similarity to native articular surface (see e.g., FIG. 7D) than TGFβ3-free sample (see e.g., FIG. 7B). The newly formed articular cartilage extended above the superior surface of the micropores and microchannels (see e.g., FIG. 7E; c.f. FIG. 7D).

Microscopically, safranin O (SO), a cationic dye that is conventionally used to label chondroitin sulfate and keratin sulfate proteoglycans that are characteristic of native articular cartilage, was positive in articular cartilage-like tissue in PCL-HA scaffold-only sample (see e.g., FIG. 7E, F). For scaffold only group without TGFβ3 delivery, SO-positive chondrocyte-like cells clustered with moderately intense SO staining of the intercellular matrix (see e.g., FIG. 7F). In contrast, regenerating articular cartilage in TGFβ3-delivery scaffold group, as exemplified by the representative sample (see e.g., FIG. 7G, H), was notably more substantial than TGFβ3-free, scaffold only sample (see e.g., FIG. 7E, F). SO staining was intense for both pericellular matrix and intercellular matrix in the TGFβ3-delivery sample (see e.g., FIG. 2G, H).

Remarkably, delivered TGFβ3 in microchannels of PLC-HA scaffold led to thoroughly distributed chondrocyte-like cells without chondrocyte clustering and notably intense SO staining (see e.g., FIG. 7G, H). Importantly, the newly formed articular cartilage extended above the superior surface of the bioengineered strands and microchannels in both TGFβ3-free and TGFβ3-delivery samples (see e.g., FIG. 7E, H). Quantitatively, TGFβ3 delivery group had significantly greater cartilage density (see e.g., FIG. 7I; n=8 per group, p=0.0001) and cartilage thickness (see e.g., FIG. 7J; n=8 per group; p=0.033) than TGFβ3-free group.

Given that no cells were transplanted in either scaffold alone or TGFβ3 delivery group, the regenerated articular cartilage must be host-derived.

Immunofluorescence of type II collagen and aggrecan was performed as follows. Expressions of collagen type II (Col-II) and aggrecan (AGC) on the articular surface of the bioengineered condylar grafts were observed using infrared imaging (Odyssey®; LI-COR, Lincoln, Nebr.). Briefly, the harvested tissue sample was bisected sagittally, washed with 0.1% Triton-X, and incubated with monoclonal antibodies: Col-II (ab7778, Abcam, Cambridge, Mass.) or AGC (ab3773; Abcam, Cambridge, Mass.) for 1.5 hrs at room temperature. Prior to incubate with AGC antibody, samples were treated with chondroitinase ABC, keratanase and keratanase II for 1 hr. Secondary antibodies conjugated with infrared fluorepores, Alexa Fluor® 680 (Invitrogen, Carlsbad, Calif.) and IRDye® 800CW (LI-COR, Lincoln, Nebr.), were diluted in 1:100 and added. Upon 1 hr incubation and washing with 0.1% Tween-20 (Sigma, St. Louis, Mo.), samples were scanned using Odyssey with 700 nm and 800 nm excitation/emission wave lengths. Immnureactivities of Col-II and AGC on both articular surface and sagittal sections were quantified using Odyssey Software. Integrated intensity of fluorescence per area was calculated as the relative quantity of immunoreactivity. Quantitative data were treated with One-way ANOVA with post-hoc LSD tests to determine differences among native cartilage, bioengineered cartilage without TGFβ3 (n=13), and with TGFβ3 (n=10).

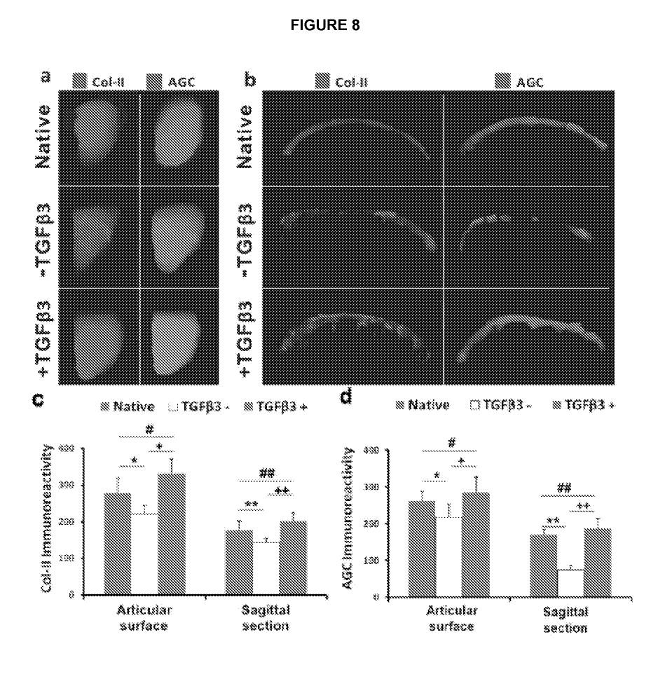

Immunoblotting results showed that type II collagen and aggrecan were evenly distributed in native rabbit humeral articular cartilage (see e.g., left column for type II collagen and right column for aggrecan in FIG. 8A for articular surface, and FIG. 8B for sagittal plane). Whereas a representative sample from TGFβ3-free scaffold group showed uneven and somewhat modest /aggrecan and type II collagen expression (see e.g., FIG. 8A, B), the representative sample of the TGFβ3-delivery group demonstrated more consistent and continuous distribution of both aggrecan and type II collagen (see e.g., FIG. 8A, B). These qualitative observations are confirmed by immunoreactivity of type II collagen and aggrecan in, e.g., FIG. 8C and FIG. 8D, respectively. Quantitatively, a representative TGFβ3-free, scaffold alone sample showed uneven and modest Col-11 andAGC (see e.g., FIG. 8C, D). In contrast, a representative TGFβ3-delivered sample showed more uniform and robust Col-11 and AGC (see e.g., FIG. 8C, D) (p=0.0001), in par or more significant than native articular cartilage. Immunoreactivity of type II collagen and aggrecan in TGFβ3-delivery group was significantly higher than TGFβ3-free scaffold group. Interestingly, immunoreactivity of type II collagen and aggrecan in TGFβ3-delivery group was also significantly higher than that of native cartilage group (see e.g., FIG. 8C, D), suggesting that the bioengineered cartilage was undergoing substantial growth.

The opposing articular surface of glenoid fossa was specifically examined for any sign of osteoarthritis, given that the bioengineered scaffold was designed from an approximately age-matched rabbit, but not specific to each rabbit that received replacement humeral joint. No appreciable sign of osteoarthritis or other cartilage injury was found in gross images (data not shown), and microscopic images with or with TGFβ3 delivery (data not shown).

### Example 9

### Formation of Vascularized Subchondral Bone in Bioengineered Joint

Anatomically shaped joint constructs, surgical joint replacement, and analysis were as described in Examples 6-8, except as otherwise noted.

Results showed that bioengineered subchondral bone integrates to bioengineered articular cartilage and host bone (see e.g., FIG. 9). A radiolucent region in proximal humeral joint cavity was present following excision of the proximal humeral head and immediately upon implantation of bioengineered scaffold (see e.g., FIG. 9A). By 8 and 16 weeks post-op, a convex, radio-opaque spheroid-shaped structure was present in the same rabbit that had received the anatomically shaped PCL-HA scaffold (see e.g., FIG. 9B, C, respectively), indicating mineralization of the bioengineered scaffold by the host. The bioengineered articular cartilage is integrated to subchondral bone (see e.g., FIG. 9D), which consisted of bone trabecula-like structures (see e.g., FIG. 9E). Von kossa staining indicates mineral deposition in microchannels (see e.g., FIG. 9F) that extends below the cartilage region (see e.g., FIG. 9C, or light to medium grey in FIG. 9F) longitudinally in microchannels. Mineral apposition was further confirmed on the surface of PCL-HA that formed the wall of interconnecting microchannels (see e.g., FIG. 9G). Bone trabeculae were populated by columnar shaped osteoblast-like cells (see e.g., FIG. 9H). The bioengineered subchondral bone was integrated to native humeral bone (see e.g., FIG. 9I), showing PCL-HA in the bioengineered bone above the dashed line, whereas native bone trabeculae, devoid of PCC-HA, below the dashed line. Multiple blood vessels were presents within microchannels formed by PCL-/HA scaffold (10.4±4.5/mm²) (see e.g., FIG. 9J, K). Average diameter of vessels was 67.11+/−28.35 μm. There were no significant differences in diameter and number of the blood vessels between TGFβ3-loaded and TGFβ3-free samples (n=10, p=0.206).

Given that no cells were transplanted in any of the groups in the above study, all blood vessels, including those exemplified in FIG. 9J and K, must have been host-derived.

1-44. (canceled)

45. A tissue module comprising:
a biocompatible matrix comprising at least two layers, a first matrix layer and a second matrix layer;
a first type of progenitor cells; and
a second type of progenitor cells;
wherein,
the first matrix layer comprises a first plurality of internal microchannels with a first average diameter and, optionally, a first plurality of pores;
the second matrix layer comprises a second plurality of internal microchannels with a second average diameter and, optionally, a second plurality of pores;
the first matrix layer comprises the first type of progenitor cells; and
the second matrix layer comprises the second type of progenitor cells.

46. The tissue module of claim 45, wherein the biocompatible matrix is an anatomically-shaped 3D composite biocompatible matrix comprising a plurality of interlaid strands forming internal microchannels.

47. The tissue module of claim 45, wherein the second matrix layer surrounds, at least in part, the first matrix layer.

48. The tissue module of claim 45, wherein the first plurality of internal microchannels and the second plurality of internal microchannels have an average diameter of about 100 μm to about 600 μm.

49. The tissue module of claim 45, wherein
the first plurality of internal microchannels have a first average diameter of about 100 μm to about 400 μm;
the second plurality of internal microchannels have a second average diameter of about 200 μm to about 600 μm; and
the first average diameter of the first plurality of internal microchannels is less than the second average diameter of the second plurality of internal microchannels.

50. The tissue module of claim 45, wherein
the first plurality of pores or the second plurality of pores are present; and
the first plurality of pores or the second plurality of pores have an average diameter of about 100 μm to about 600 μm.

51. The tissue module of claim 45, wherein the first matrix layer or the second matrix layer comprise at least one material independently selected from the group consisting of fibrin, fibrinogen, a collagen, a polyorthoester, a polyvinyl alcohol, a polyamide, a polycarbonate, a polyvinyl pyrrolidone, a marine adhesive protein, a cyanoacrylate, a polymeric hydrogel, and an inorganic mineral, or a combination thereof.

52. The tissue module of claim 51, wherein the first matrix layer or the second matrix layer comprise polycaprolactone and hydroxyapatite.

53. The tissue module of claim 51, wherein the first matrix layer comprises polycaprolactone and the second matrix layer comprises polyethylene glycol hydrogel.

54. The tissue module of claim 45, wherein the first type of progenitor cells are bone progenitor cells selected from the group consisting of mesenchymal stem cells (MSC), MSC-derived cells, and osteoblasts, or a combination thereof.

55. The tissue module of claim 45, wherein the second type of progenitor cells are cartilage progenitor cells selected from the group consisting of mesenchymal stem cells (MSC), MSC-derived cells, and chondrocytes, or a combination thereof.

56. The tissue module of claim 45, wherein the tissue module comprises progenitor cells at a density of at least about 0.0001 million cells (M) ml⁻¹ up to about 1000 M ml⁻¹.

57. The tissue module of claim 45, wherein the ratio of the first type of progenitor cells to the second type of progenitor cells is from at least about 100:1 up to about 1:100.

58. The tissue module of claim 45, wherein the first matrix layer or the second matrix layer further comprise at least one agent selected from the group consisting of a bioactive molecule, biologic drug, diagnostic agent, or strengthening agent; or the step of introducing an agent selected from the group consisting of a bioactive molecule, biologic drug, diagnostic agent, and strengthening agent to the matrix material, or a combination thereof.

59. The tissue module of claim 58, wherein the first matrix layer or the second matrix layer comprise at least one agent independently selected from the group consisting of an osteoinductive cytokine and a chondroinductive cytokine.

60. The tissue module of claim 59, wherein the first matrix layer or the second matrix layer comprise at least one agent independently selected from the group consisting of TGFβ, bFGF, VEGF, and PDGF, or a combination thereof.

61. The tissue module of claim 45, wherein the biocompatible matrix has a 3D anatomical shape selected from the group consisting of a fibrous joint, a cartilaginous joint, or a synovial joint.

62. The tissue module of claim 61, wherein the biocompatible matrix has a 3D anatomical shape of a synovial joint selected from the group consisting of a ball and socket joint, condyloid joint, saddle joint, hinge joint, pivot joint, and gliding joint.

63. The tissue module of claim 61, wherein the biocompatible matrix has a 3D anatomical shape of a synovial joint selected from the group consisting of a proximal tibial condyle, proximal humeral condyle, femoral condyle, and mandibular condyle.

64. A tissue module comprising:
(i) a first biocompatible matrix layer comprising
(a) polycaprolactone and hydroxyapatite;
(b) bone progenitor cells selected from the group consisting of mesenchymal stem cells (MSC), MSC-derived cells, and osteoblasts, or a combination thereof;
(c) an osteoinductive cytokine;
(d) a first plurality of internal microchannels having a first average diameter of about 200 μm; and
(e) a first plurality of pores having an average diameter of 400 μm;
(ii) a second biocompatible matrix layer comprising
(a) polyethylene glycol hydrogel;
(b) cartilage progenitor cells selected from the group consisting of mesenchymal stem cells (MSC), MSC-derived cells, and chondrocytes, or a combination thereof;
(c) a chondroinductive cytokine; and
(d) a second plurality of internal microchannels having a second average diameter of about 400 μm;
wherein,
the second matrix layer surrounds, at least in part, the first matrix layer;
bone progenitor cells and cartilage progenitor cells are present at a total average density of at least about 0.0001 million cells (M) ml⁻¹ up to about 1000 M ml⁻¹;
the ratio of the bone progenitor cells to the cartilage progenitor cells is from at least about 20:1 up to about 1:20; and
the tissue module has a 3D anatomical shape selected from the group consisting of a fibrous joint, a cartilaginous joint, or a synovial joint.

65. A method of treating a tissue defect in a subject comprising:
grafting the tissue module of claim 45 into a subject in need thereof;
wherein, the tissue defect is associated with arthritis; osteoarthritis; osteoporosis; osteochondrosis; osteochondritis; osteogenesis imperfecta; osteomyelitis; osteophytes; achondroplasia; costochondritis; chondroma; chondrosarcoma; herniated disk; Klippel-Feil syndrome; osteitis deformans; osteitis fibrosa cystica, a congenital defect resulting in absence of a tissue; accidental tissue defect; fracture; wound; joint trauma; an autoimmune disorder; diabetes; cancer; a disease, disorder, or condition that requires the removal of a tissue; or a disease, disorder, or condition that affects the trabecular to cortical bone ratio.
